(19) United States
(12) Patent Application Publication
(10) Pub. No.: US 2025/0213614 A1
(43) Pub. Date: Jul. 3, 2025

(54) Modified RELA Protein for Inducing Interferon Expression and Engineered Immune Cells with Improved Interferon Expression

(71) Applicants: Institut Curie, Paris (FR); Institut National de la Santé et de la Recherche Médicale, Paris (FR)

(72) Inventors: Nicolas Manel, Paris (FR); Nadia Jeremiah, Issy les Moulineaux (FR)

(73) Assignees: Institut Curie, Paris (FR); Institut National de la Santé et de la Recherche Médicale, Paris (FR)

(21) Appl. No.: 18/851,221

(22) PCT Filed: Mar. 30, 2023
(86) PCT No.: PCT/EP2023/058224

§ 371 Date: Sep. 26, 2024

(30) Foreign Application Priority Data
Mar. 31, 2022 (EP) 22305421.4

(51) Int. Cl. A61K 35/17; A61K 38/00; A61K 40/11; A61K 40/31; A61K 40/42; A61P 31/18; A61P 35/00; C07K 14/47; C07K 14/555; C12N 5/0783; C12N 15/85

(52) U.S. Cl. CPC A61K 35/17; A61K 40/11; A61K 40/31; A61K 40/4238; A61P 31/18; A61P 35/00; C07K 14/4705; C07K 14/555; C12N 5/0636; C12N 15/85; A61K 38/00; A61K 2239/15; C12N 2501/72; C12N 2510/00

(57) ABSTRACT

The invention is in the field of immunotherapy. The present application provides modified RELA protein which are useful for inducing or promoting interferon expression by immune cells, in particular T cells. The invention enables the production of immune cells with an activated or enhanced interferon metabolism. The present application also relates to immune cells, in particular T cells, comprising and/or expressing a modified RELA protein according to the invention, such cells having an activated interferon metabolism. The present invention also provides in vitro and/or ex vivo method of preparing immune cells, in particular T cells, useful in immunotherapy. The invention also relates to methods for treating a patient, in particular a patient who has a cancer or an infectious disease, in particular an infection by a virus.

### TECHNICAL FIELD OF THE INVENTION

The invention is in the field of immunotherapy. The present application provides modified RELA protein which are useful for inducing or promoting interferon expression by immune cells, in particular T cells. The invention enables the production of immune cells with an activated or enhanced interferon metabolism. The present application also relates to immune cells, in particular T cells, comprising a modified RELA protein according to the invention, such cells having an activated interferon metabolism. The present invention also provides in vitro and/or ex vivo method of preparing immune cells, in particular T cells, useful in immunotherapy. The invention also relates to methods for treating patient, in particular patient who has a cancer or an infectious disease, in particular an infection by a virus. The invention is also related to immune cells and pharmaceutical compositions comprising immune cells prepared according to a method of the invention and/or having a modified RELA protein according to the invention.

### BACKGROUND OF THE INVENTION

The classic paradigm of immunology describes a division of labour between the innate and adaptive immune cell types¹. Innate immune cells armed with germ-line encoded pathogen recognition receptors (PRR) sense the presence of pathogens or danger and respond by producing cytokines, upregulating co-stimulatory molecules and presenting antigens. Adaptive immune cells are subsequently clonally stimulated by antigenic peptide presentation to mediate helper and effector functions. Even though it is now recognized that PRR are also expressed in adaptive cells, these cells are not generally recognized for having the capacity to efficiently detect and signal pathogens or danger the way innate immune cells do. The mechanisms that differentiate innate and adaptive cells downstream of PRR are elusive.

Type I and type III interferons (IFN) are crucial cytokines that activate antiviral defences and contribute to inflammation downstream of PRR. IFN-I/III expression is controlled by the transcription factors NF-κB, IRF3, IRF7, ATF2 and c-Jun, which are tightly controlled by multiple post-translational modifications (PTM) to ensure directed and timely expression of target genes. Detection of intracellular nucleic acids by PRRs—dsRNA by RIG-1 and MDA5, dsDNA by the Cyclic GMP-AMP synthase cGAS-cGAMP-STING pathway—potently stimulates IFN-I/III expression. IFN-I is also expressed constitutively at low levels, resulting in tonic IFN signaling that is crucial for antiviral defences^{2,3}. cGAS activation by endogenous DNA has been implicated in constitutive IFN production^{4,5}. RELA (p65 subunit of NF-κB) is also required for autocrine IFN-I production at baseline ⁶.

While antigen-presenting cells readily produce IFN-I/IIII in response to PRR stimulation, lymphocytes are not generally considered a significant source of IFN-I/III. For example, CD4+ T cells are not able to produce IFN-I in response to HIV infection, resulting in their inability to control virus infection, while monocyte-derived dendritic cells do so in similar conditions^{7,8}. However, IFN production by T cells has been associated with desirable functional outcomes, including spontaneous resistance to HIV infection 9 and anti-tumor activity of chimeric antigen receptor (CAR) T cells¹⁰.

T cells express a wide range of interferon-inducing PRR at the protein level, including cGAS, STING and RIG-I^{11,12}. Infection with Sendai or HIV viruses that activates RIG-1 leads to detectable levels of IFN-I in T cells^{7,12}. In one study, IFN-I production has been detected following cGAS-STING stimulation by electroporation of DNA or cGAMP, or following infection with a Herpes Simplex Virus type 1 mutant 8, while another study failed to detect IFN-I production and IFN responses after DNA transfection¹³. IFN production by T cells has also been observed in pathogenic conditions. Splenic T cells with a pathogenic mutation in TREX1 produce elevated levels of IFN-I¹⁴. IFN-I expression in T cells has also been described in the context of cancer but the upstream signaling was not evaluated^{15,16}. PRR activation in T cells additionally leads to IFN-independent responses in T cells. Endogenous cGAS-STING driven IFN-I signaling contributes to stemness maintenance in CD8+ T cells in the context of cancer immune responses¹⁷, while STING activation by exogenous agonists leads to cell death in murine T cells^{11,18,19}. There exist important species-specific differences in activation and response to STING signaling between murine and human T cells. DMXAA, a STING agonist with potent anti-tumor function failed in clinical trials due to sequence differences between human and murine STING²⁰. Additionally, pathogenic variants in STING lead to T cell imbalances in peripheral naive and memory compartments and a block in proliferation in humans whereas the mice model develops a SCID phenotype^{21, 22, 23, 24, 25}. Individually, these findings illustrate the notion that CD4+ T cells have the capacity to produce IFN-I/III, but a general mechanism to explain the control of IFN-I/III expression is lacking. As a result, this ability has yet to be exploited for functional applications.

### SUMMARY OF THE INVENTION

Although clinical benefits have been observed in treatment of several diseases, including cancers, the efficacy of immune cells therapy in therapies is far limited since transferred immune cells occasionally fail to exhibit the desired functions and/or survival in vivo. Clinical benefits have been obtained in treatment of infection, for example by administering T cells to a patient. However, due to poor functionality and persistence of transferred T cells, and/or due to adverse effects in vivo, the efficacy of immune cell therapies in the treatment of several disease needs to be improved. Therefore, new, and possibly complementary, engineering of immune cells may help to improve immune cells function and survival in vivo. The use of genetically modified immune cells, like modified T cells, in therapies suffers from several drawbacks and hurdles, therefore limiting their use despite their attractive potential.

To address, at least partially, at least one problem of the state of the art, it is an object of the invention to provide means for modifying immune cell(s) that may overcome the drawbacks of the prior art. The present inventors found that modified RELA protein (also known as transcription factor p65, nuclear factor NF-kappa-B p65 subunit, and p65) have the capability to induce expression of interferon by immune cells. In particular, RELA protein with substituted lysine amino acid residue(s) as compared to wild-type RELA protein enhances the expression of interferon (IFN), in particular IFN-I and/or IFN-III, by T cells. The author further demonstrated that different types of modified RELA proteins were able to enhance the expression of IFN by immune cells.

The present inventors also showed for the first time that engineered immune cells, and in particular T cells, with modified RELA protein by substitution of one or several lysine amino acid residue(s) as compared to RELA wild-type protein have an enhanced resistance towards infection, in particular viral infection, as compared to non-engineered immune cells.

Further, the inventors provide evidence that engineered immune cells, in particular T cells or CAR-T cells, with a modified RELA protein according to the invention have an increased anti-tumor activity as compared to non-engineered immune cells.

The invention more particularly provides a novel strategy for improving at least one capability of immune cells, in particular of T cells, more particularly CD4+ T cells, CD8+ T cells and CAR-T cells, by allowing genetically engineered T cells to produce interferon. Without the need to rely on extracellular source of interferon, the metabolism of engineered T cells allows them to have prolonged survival and/or enhanced proliferation capability and/or improved cytotoxicity, in particular in vivo, thereby enhancing overall T cell response for treating a disease or a condition, in particular against infection, including viral infection, or cancer.

The invention also relates to methods for treating patient, in particular patient who has a cancer or an infectious disease. The invention is also related to T cell(s) and pharmaceutical compositions comprising T cell(s) prepared according to a method of the invention.

Conferring to immune cells, in particular T cells, the capability to autonomously produce interferon provides a crucial advantage against immunosuppressive mechanisms, particularly in the tumor or infectious environments. Immune cell-intrinsic interferon production may foster a relatively rapid and efficient anti-tumor immune response or anti-infectious response. It was not shown or suggested that modifying RELA protein would give the capability to immune cells to produce interferon, and that this production would be sufficient to sustain, restore or activate immune cells function(s), in particular enhanced survival and/or improved cytotoxicity that may be triggered against disease cells such as tumor cells or infected cells in a safely manner. Thereby, the authors of the invention provide immune cells, and method for engineering immune cells with a metabolic advantage. By conferring to immune cells the capability to produce interferon, the inventors surprisingly show that this capability is sufficient to exhibit cytotoxic activity against tumor cells, exhibit resistance against infection, in particular by a virus, and more particularly resistance against HIV infection.

### SHORT DESCRIPTION OF THE DRAWINGS

These and further aspects of the invention will be explained in greater detail by way of examples and with reference to the accompanying drawings in which:

FIG. 1. IFN-I/III production is stunted in CD4+ T cells compared to dendritic cells.

(A) Stimulation with extracellular STING ligands in primary blood cells, experimental outline. (B) IFN-I/III concentrations after treatment of enriched total resting CD4+T subsets and FACS-sorted pDC, cDC1, cDC2 with cGAMP or ADU-S100 (1 μg or 2.5 μg) (n=3-4 donors combined from 2 independent experiments). (C) Western blot of key signaling proteins involved in STING signaling and control proteins in the indicated resting primary cell types (representative of n=3 independent experiments). (D) IFN-I/III concentrations after stimulation of TCR-activated CD4+ T cells (CD4T) and MDDC with ADU-S100 (2.5 μg) stimulation (n=8 donors combined from 4 independent experiments). (E) Delivery of STING ligands by permeabilization, experimental design. (F) IFN-I/III concentrations after treatment of TCR-activated CD4+ T cells and MDDC with cGAMP (0.06, 0.6 and 6 μg/ml) delivered by digitonin-mediated membrane permeabilization (n=10 donors combined from 5 independent experiments). (G) Western blot of key signaling proteins involved in STING signaling and control proteins 6 hours following cGAMP (6 μg/ml) stimulation in TCR-activated CD4+ T cells and MDDC (representative of n=3 independent experiments). (H) IFN-I concentrations after stimulation of TCR-activated CD4+T and MDDC with cGAMP (0.06, 6 μg/ml). Cells were conditioned for 24 hours prior to stimulation with the indicated media (CM=conditioned media) or treated with recombinant IFNγ (10 ng/ml) (n=4 donors from 2 independent experiments). (I) IFN-I/III concentrations after infection of TCR-activated CD4+ T cells and MDDC with Sendai virus (SeV, 200 HA/ml) (n=9 donors combined from 5 independent experiments). Each symbol represents one donor, bars represent geometric mean, paired one-way ANOVA with Tukey multiple comparison test.

FIG. 2. RELA enhances IFN-I/III expression in CD4+ T cells

(A) STING ligand stimulation in cells transduced with RELA, experimental outline. (B) IFN-I/III concentration following cGAMP (6 μg/ml) stimulation of TCR-activated CD4+ T cells and MDDC transduced with control (GFP) or RELA lentivectors (n=8 donors combined from 4 individual experiments). (C) Western blot of RELA and actin in CD4+ T cells and MDDC transduced with either control, RELA, RELA K5Q, or RELA K5R (representative of n=2 independent experiments). (D) IFN-I/III concentration after cGAMP (6 μg/ml) stimulation of CD4+T and MDDC transduced with control (GFP), RELA, RELA K5Q or RELA K5R (n=4 donors combined from 2 individual experiments). (E) Western blot of RELA and actin four days after lentiviral transduction of CD4+ T cells as indicated (representative of n=2 independent experiments). (F) IFN-I/III concentration following cGAMP (0.06, 6 μg/ml) stimulation of CD4+ T cells transduced with control (GFP), RELA, RELA K310R or RELA K5R (n=4 donors combined from 2 independent experiments). Each symbol represents one donor, bars represent geometric mean, paired one-way ANOVA with Tukey's multiple comparison test.

FIG. 3. IRF3 and a DNA methylation inhibitor synergize with RELA to fully lift the IFN-I/III restriction in CD4+ T cells.

(A) cGAMP stimulation in CD4+ T cells treated with a DNA methylation inhibitor (5AZA) and transduced with RELA and IRF3 lentivectors, outline of the experiment. (B) IFN-I/III concentration after cGAMP (6 μg/ml) stimulation of CD4+ T cells and MDDC transduced with either control or IRF3 (n=12 donors combined from 6 independent experiments). (C) Western blot of key signaling proteins involved in STING signaling and actin 6 hours following cGAMP (6 μg/ml) stimulation of CD4+ T cells and MDDC transduced with either control or IRF3 lentivectors (representative of 2 independent experiment). (D) Viability (mean+SEM) and IFNλ1 concentration (geometric mean) following 5AZA (2 PM) treatment and cGAMP (0.06, 6 μg/ml) stimulation of CD4+ T cells (n=6 donors from 3 independent experiments). (E) Western blot of key signaling proteins involved in STING signaling and actin 6 hours following cGAMP (6 μg/ml) stimulation of CD4+ T cells pretreated for 48 hours with 5AZA (2 μM) (representative of 2 independent experiments). (F) IFN-I/III concentration following cGAMP (6 μg/ml) stimulation of CD4+ T cells transduced with control (GFP), IRF3, RELA K5R and pretreated for 48 hours with 5AZA (2 μM) (n=8 donors combined from 4 independent experiments, geometric mean). (G) IFN-I/III concentration following cGAMP (6 μg/ml) stimulation of untransduced MDDC and CD4+ T cells transduced with control (GFP), IRF3, RELA K5R and treated for 48 hours with 5AZA (2 μM) (n=4 donors combined from 2 independent experiments). Each symbol represents one donor, bars represent geometric mean, paired one-way ANOVA with Tukey's multiple comparison test.

FIG. 4. IRF7 is required for IFN-I/III expression in iCD4+ T cells.

(A) IFN-I/III concentration following cGAMP (6 μg/ml) stimulation of CD4+ T cells transduced with control (GFP), RELA K5R, IRF3 and pretreated for 48 hours or control and 5AZA (2 μM) (n=4 donors combined from 2 independent experiments). (B) Western blot of RELA, IRF7 and actin in CD4+ T cells transduced with either LacZsh, IRF7sh1 or IRF7sh5 and treated with IFNα2a (1000 U/ml) for 18 to 24 hours (n=2, one out of 4 representative donors shown). (C) IFN-I/III concentration following cGAMP (6 μg/ml) stimulation of CD4+ T cells co-transduced with control (GFP) or RELA K5R and LacZsh, IRF7sh1 or IRF7sh5 (n=4 donors from 2 independent experiments). Each symbol represents one donor, bars represent geometric mean, paired one-way ANOVA with Tukey's multiple comparison test.

FIG. 5. Tonic cGAS activity is required for IFN expression in iCD4+ T cells.

(A) Western blot of cGAS, IRF3, IRF7, RELA and actin in CD4+ T cells nucleofected with either control or cGAS targeting guides and subsequently transduced with control (GFP), RELA K5R or IRF3 lentivectors (representative of n=2 independent experiments). (B) cGAMP quantification by ELISA in CD4+ T cells transduced with either control (GFP), RELA K5R or IRF3 lentivectors (n=4 donors combined from 2 independent experiments). (C) IFN-I/III concentration following cGAMP (6 μg/ml) stimulation of CD4+ T cells nucleofected with control or cGAS gRNA and transduced with control, RELA K5R or IRF3 lentivectors. Cells were pretreated with 5AZA (2 μM) for 48 hours prior to cGAMP stimulation (n=4 donors combined from 2 independent experiments). Each symbol represents one donor, bars represent geometric mean, paired one-way ANOVA with Tukey's multiple comparison test. (D) DAPI, cGAS staining and GFP localization by confocal microscopy in CD4+ T cells transduced with control (GFP), RELA K5R or IRF3 lentivectors. Scale bar at 10 μM. Right panel, magnification of cGAS channel showing contours used to delineate nuclear and cellular contents, scale bar at 5 μM (n=4 donors combined from 2 independent experiments). (E) Quantification of the ratio of average intensity of nuclear to cytoplasmic cGAS signal in CD4+ T cells transduced with either control (GFP), RELA K5R and IRF3 lentivectors (n=4 donors combined from 2 independent experiments). Each symbol represents one cell, bars represent median, mixed-effect analysis.

FIG. 6. iCD4+ T cells resist HIV infection and enhance CAR mediated tumor killing.

(A) HIV infection challenge in iCD4+ T cells, experimental outline. (B) Rate of HIV-1 or HIV-2 infection, 48 hours post infection of CD4+ T cells transduced with control (GFP), IRF3 and RELA K5R lentivectors. Cells were transduced, pretreated with 5AZA (2 μM) for 48 hours and subsequently infected with HIV-1 or HIV-2 single-round virus (n=4 donors combined from 2 independent experiments). Each symbol represents one donor, bars represent mean±SEM of 4 donors, paired one-way ANOVA with Tukey's multiple comparison test of highest dose of virus. (C) Pearson Correlation of infection rates with IFNλ1 concentration of CD4+ T cells transduced and treated with 5AZA as indicated. (D) CAR-T tumor spheroid killing assay, experimental outline. (E) Representative images of mKate2+CD19+A549 cells (red) alone or in co-culture with CAR+ GFP+ or CAR+ RELA K5R+ T cells (black) acquired over 5 days. (F) Fluorescence intensity of mKate2+CD19+A549 cells over time during co-culture with CAR+ GFP+ or CAR+ RELA K5R+ T cells (n=10 donors combined from 5 independent experiments). Each symbol represents mean with the shaded region representing SEM, two-way ANOVA. (G) Working model. RELA functions as rheostat to control IFN-I/III expression levels in CD4+ T cells. IFN-I/III expression requires tonic cGAS activity or PRR stimulation and positive feedback from IRF7 signaling.

### DETAILED DESCRIPTION OF SPECIFIC EMBODIMENTS OF THE INVENTION

In a first aspect, the invention relates to a modified RELA protein for modifying the metabolism of immune cells, in particular T cells. In another aspect, the invention relates to an immune cell comprising a modified RELA protein, or able to produce and or express a modified RELA protein. In another aspect, the invention relates to a modified RELA protein and immune cells expressing or comprising a modified RELA protein, for use in the treatment of a disease. Other aspects of the invention are detailed in the detailed description and in the examples of the invention.

### Immune Cells

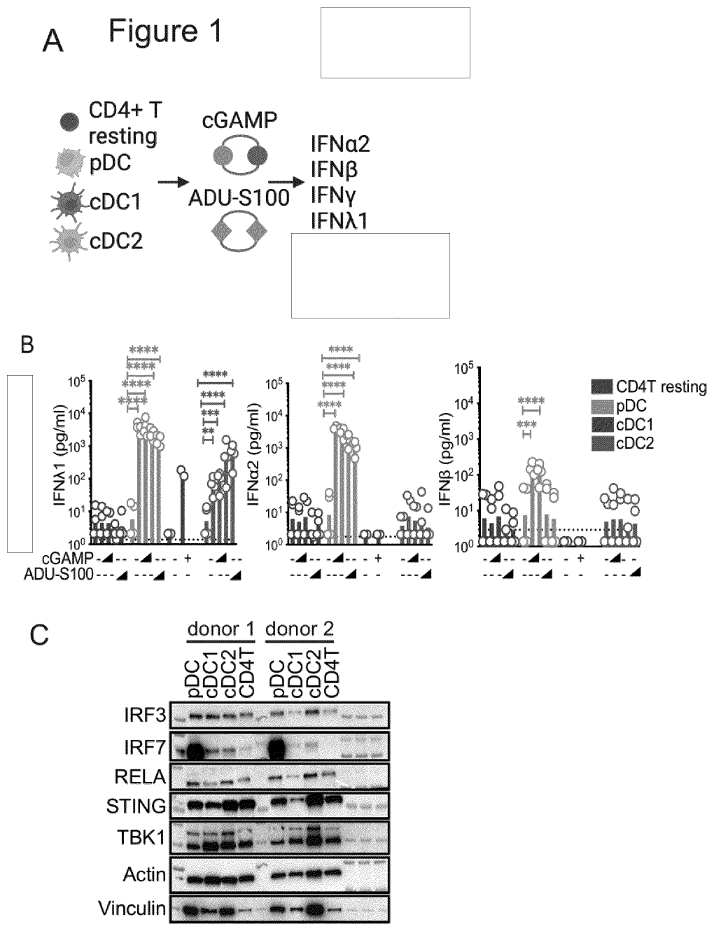

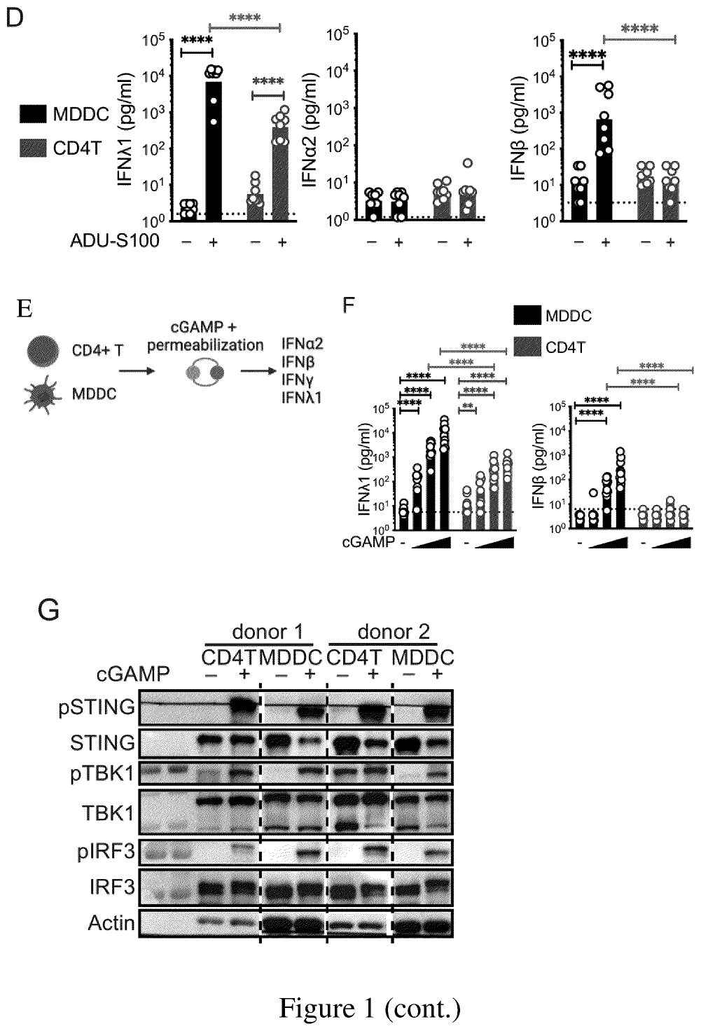

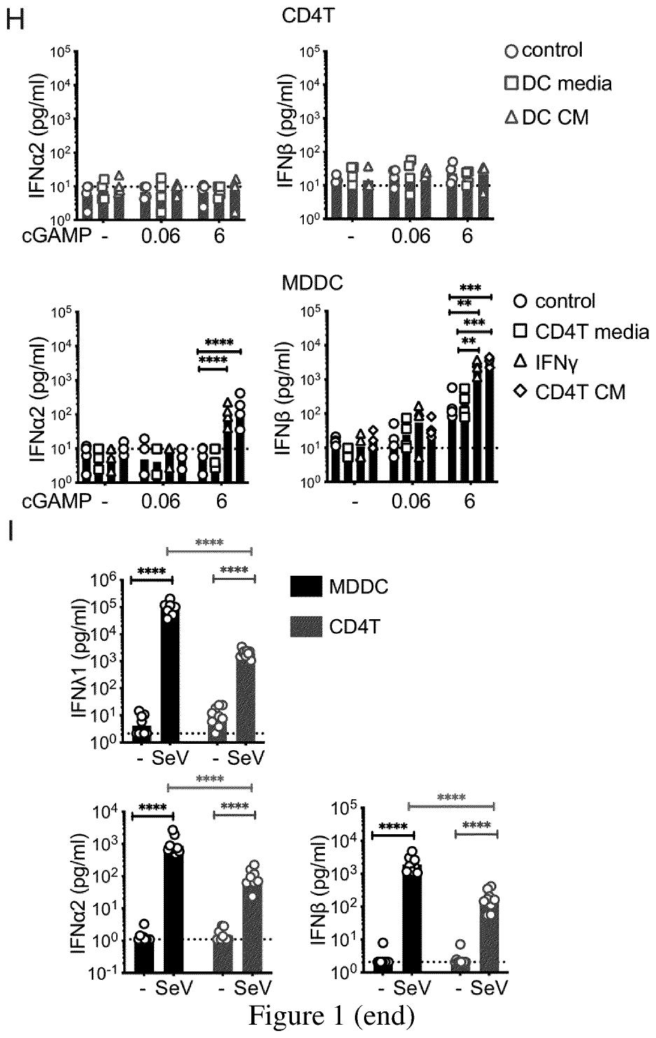

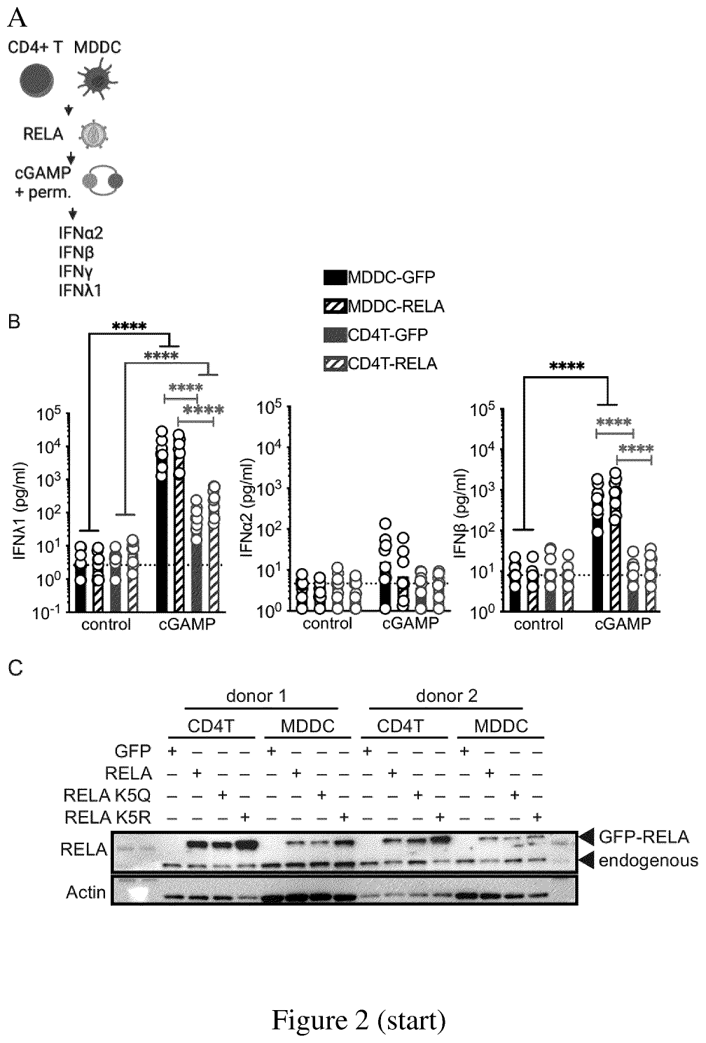

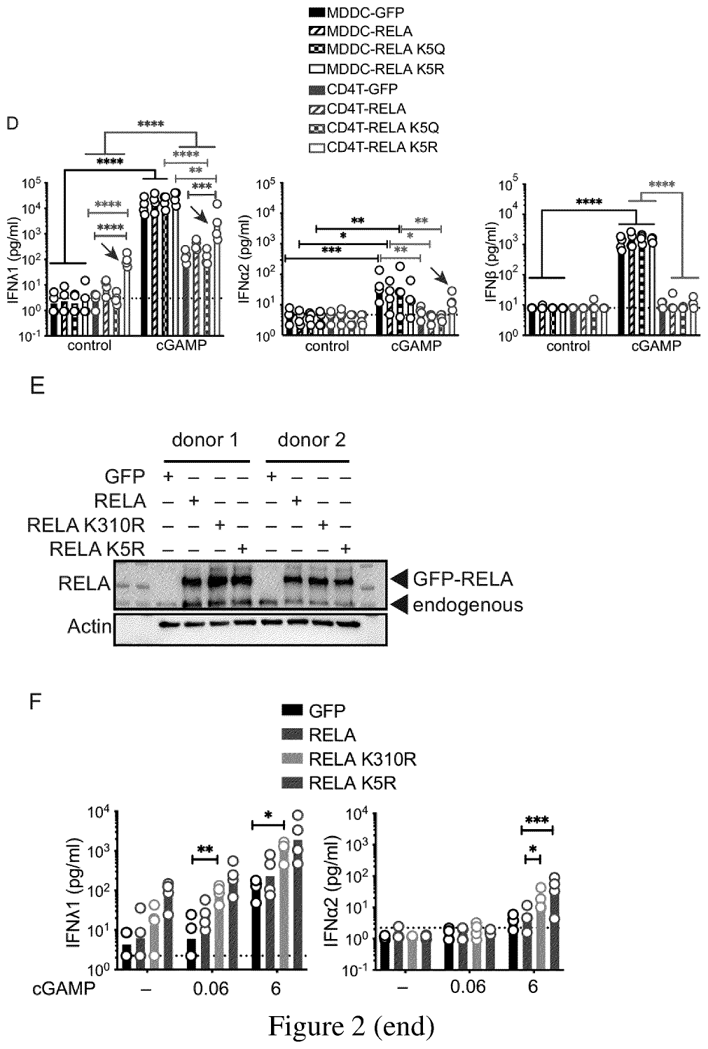

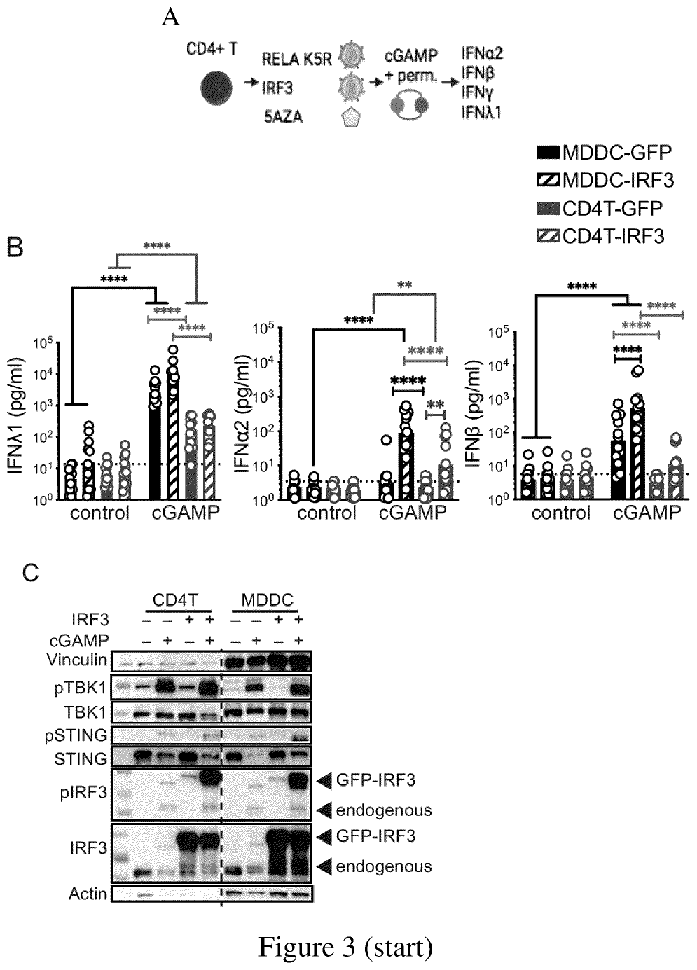

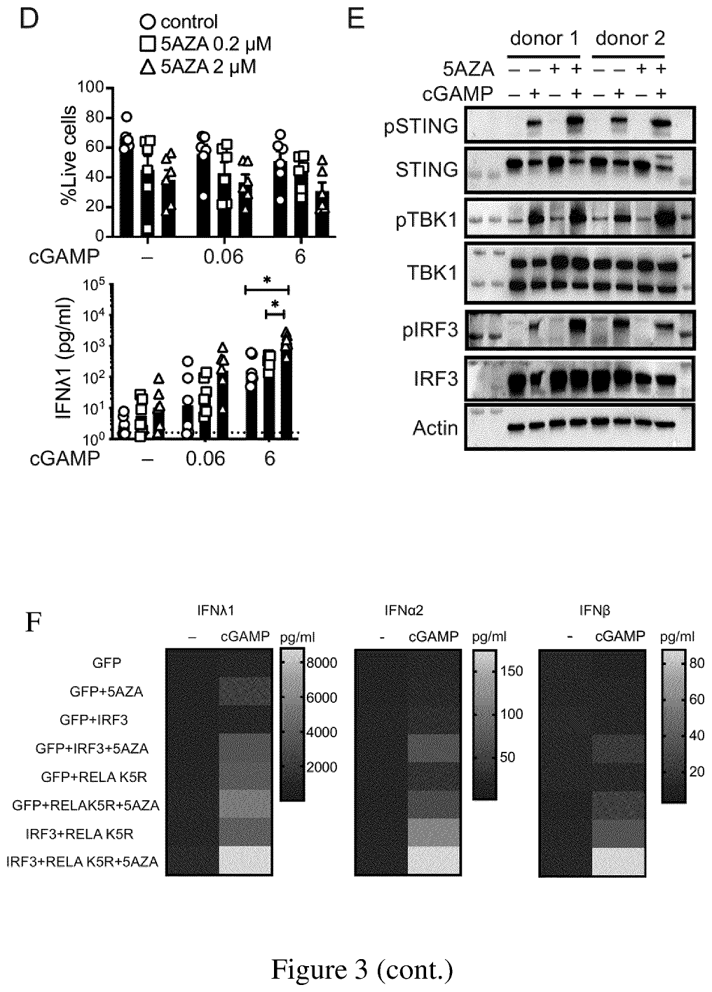

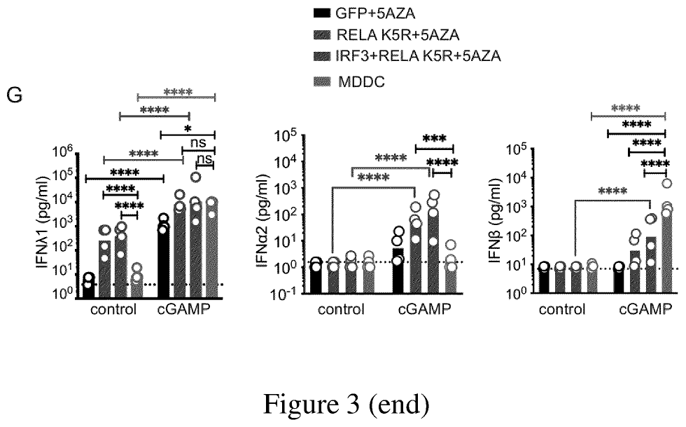

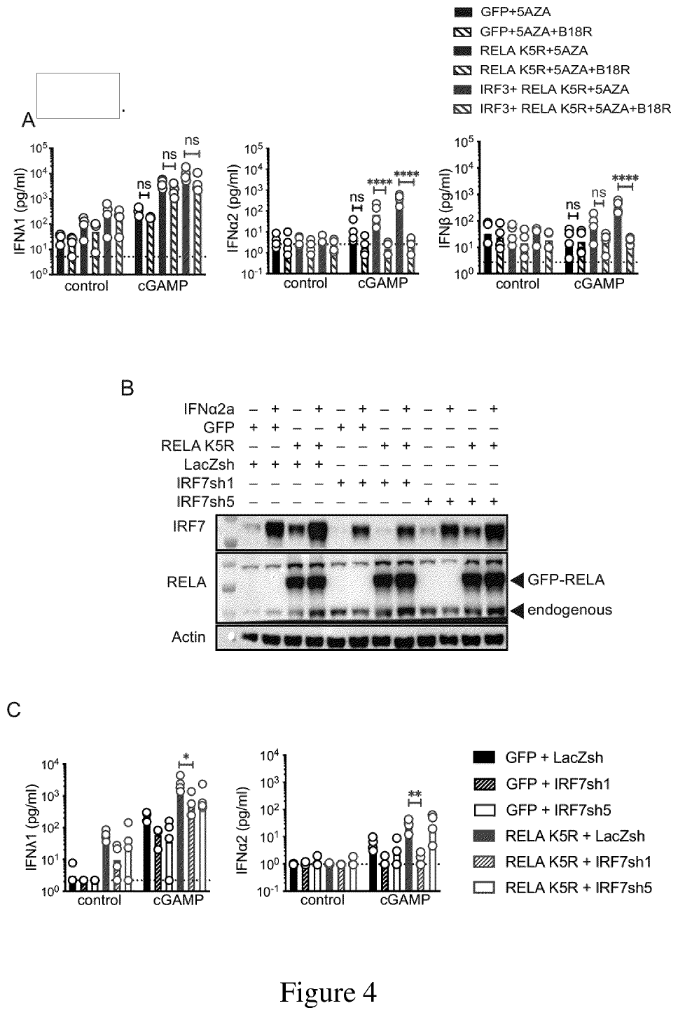

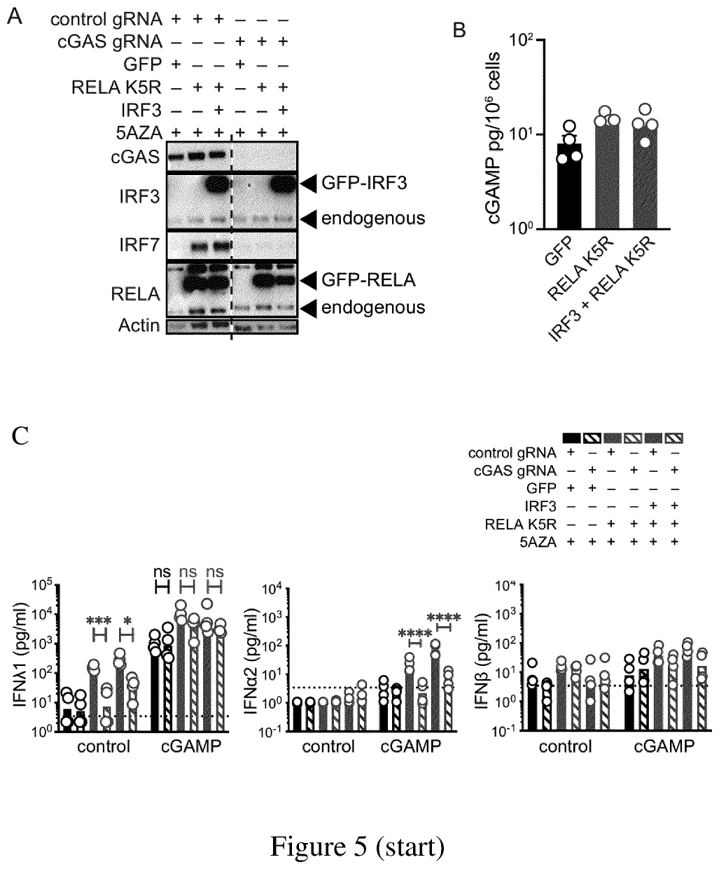

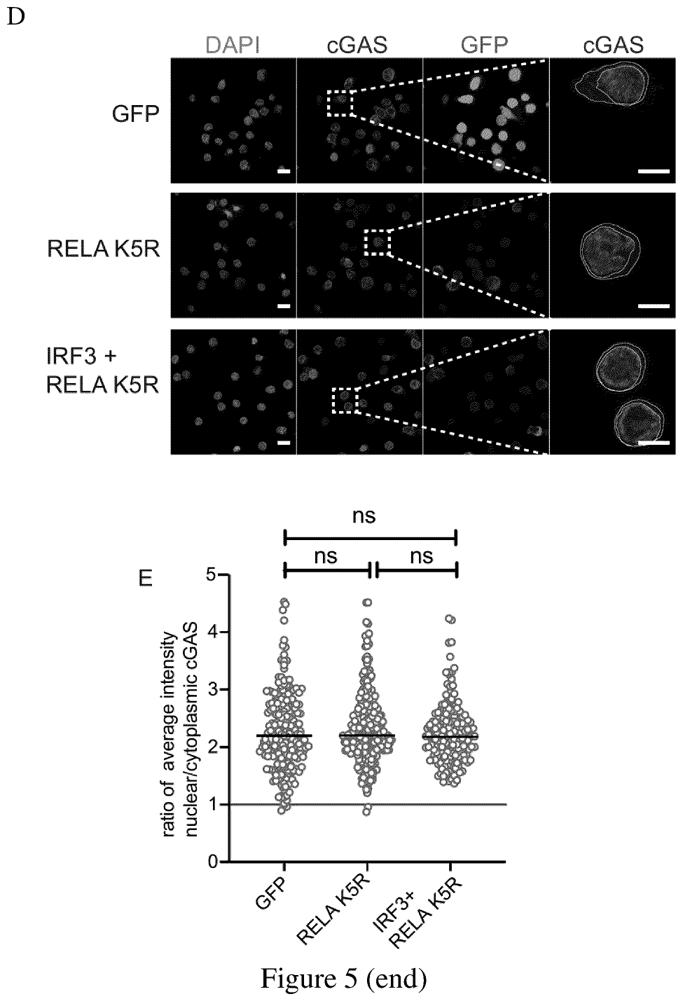

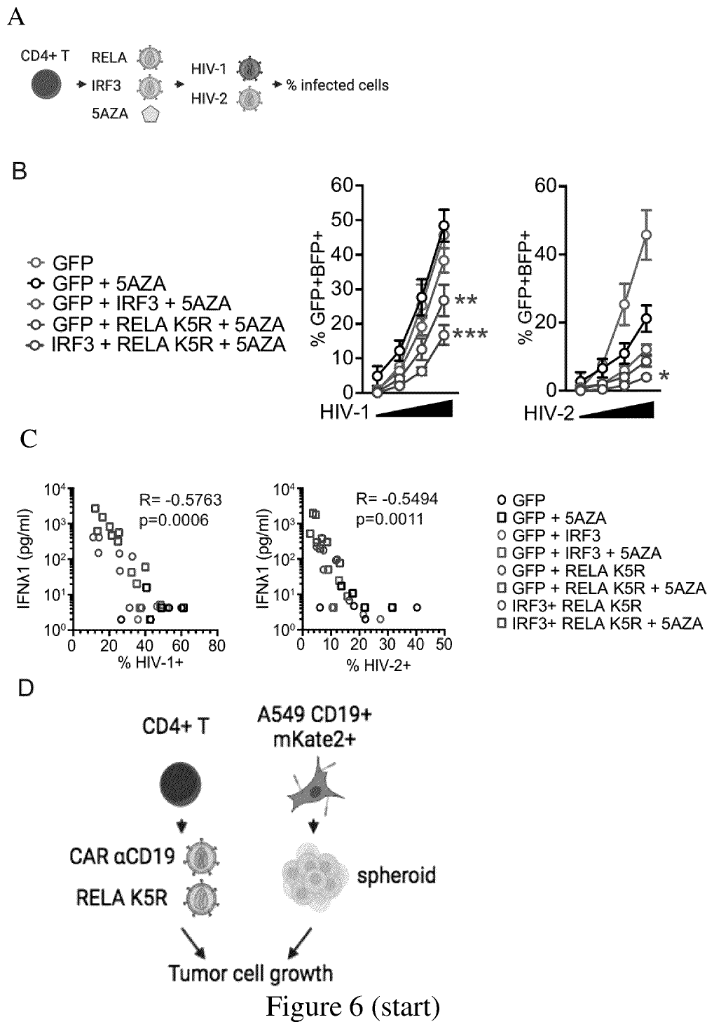

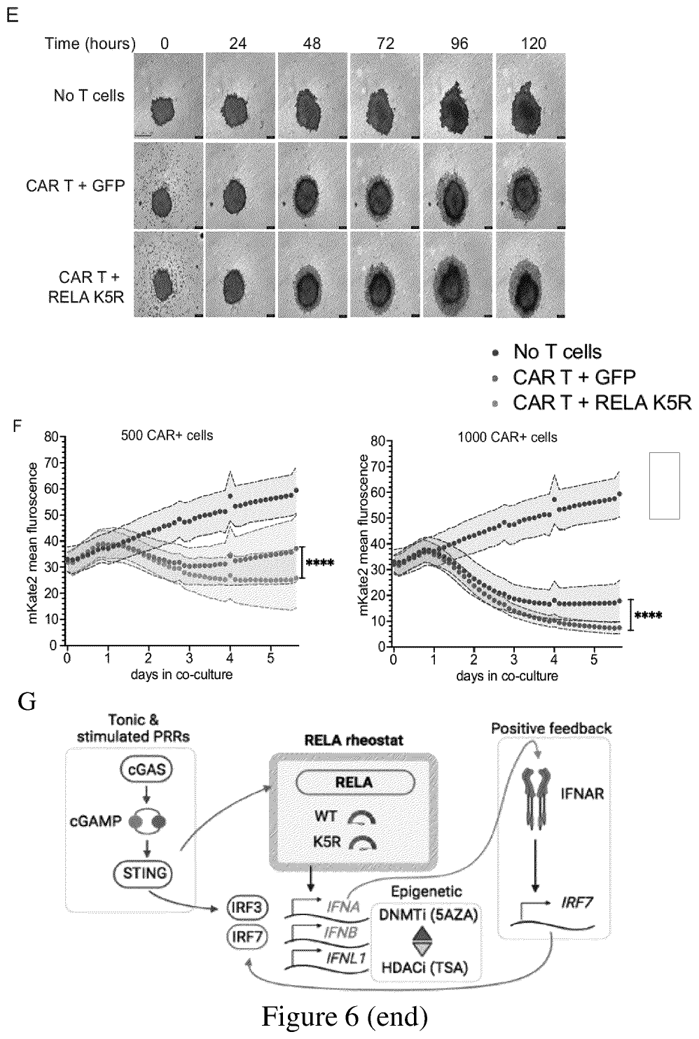

In a first aspect of the invention, it is provided engineered immune cells, in particular engineered T cells, with an improved production of interferon, in particular with an improved production of IFN-1 and/or IFN-III, as compared to an unmodified T cell. Improvement of the production of IFN in engineered immune cells may be assessed by comparison of the IFN production in engineered immune cells and in control immune cells (i.e. unmodified cells that are not stimulated for producing IFN), the engineered immune cells and the control immune cells being issued from the same type of cells, in particular from the same patient or human being. Interferon production may be assessed according to any method disclosed in the examples of the invention, in particular according to the material and method associated with the results illustrated in FIG. 1.

In an embodiment of the invention, it is provided T cells comprising and/or expressing and/or having the capability to express a modified RELA protein as disclosed herein.

In an embodiment, the modified RELA protein can bind to DNA implicated in IFN-I expression, like a wild type RELA protein. The ability to bind to DNA implicated in IFN-I expression may be assessed by methods known by the skilled artisan, for example by competition binding between a modified RELA protein and a wild type RELA protein on DNA implicated in the IFN-I expression.

In an embodiment, the modified RELA protein induces IFN-I production in immune cells, in particular in T cells.

In an embodiment, the modified RELA protein can bind to DNA implicated in IFN-I expression, like a wild type RELA protein, and the modified RELA protein induces IFN-I production in immune cells, in particular in T cells.

An immune cell according to the invention may comprise and/or express any modified RELA protein as disclosed herein, and/or may comprise any genetic construct encoding such a modified RELA protein. In particular, an immune cell according to the invention may comprise and/or express a modified RELA protein derived from a wild-type RELA protein, in particular a wild-type human RELA protein, more particularly a wild-type human RELA protein of SEQ ID No. 1, by substitution of at least one lysine residue for a non-lysine residue. The immune cell according to the invention may alternatively or complementarily comprise a genetic construct encoding a modified RELA protein derived from a wild-type RELA protein, in particular a wild-type human RELA protein, more particularly a wild-type human RELA protein of SEQ ID No. 1, by substitution of at least one lysine residue for a non-lysine residue.

An immune cell according to the invention may comprise and/or express a modified RELA protein and/or may comprise a genetic construct encoding a modified RELA protein wherein the at least one substituted lysine residue is localized at position 122, 123, 310, 314 or 315, more particularly at position 310, of the wild-type RELA protein, in particular the wild-type RELA protein of the sequence of SEQ ID No. 1.

In an aspect of the invention, an immune cell according to the invention may comprise and/or express a modified RELA protein derived or issued from a wild-type RELA protein, in particular a wild-type human RELA protein, more particularly a wild-type human RELA protein of SEQ ID No. 1, and/or may comprise a genetic construct encoding such a modified RELA protein, wherein the lysine localized at position 122 is substituted for a non-lysine amino acid residue, as compared to the wild type RELA protein of SEQ ID No. 1.

In an aspect of the invention, an immune cell according to the invention may comprise and/or express a modified RELA protein derived or issued from a wild-type RELA protein, in particular a wild-type human RELA protein, more particularly a wild-type human RELA protein of SEQ ID No. 1, and/or may comprise a genetic construct encoding such a modified RELA protein, wherein the lysine localized at position 123 is substituted for a non-lysine amino acid residue, as compared to the wild type RELA protein of SEQ ID No. 1.

In an aspect of the invention, an immune cell according to the invention may comprise and/or express a modified RELA protein derived or issued from a wild-type RELA protein, in particular a wild-type human RELA protein, more particularly a wild-type human RELA protein of SEQ ID No. 1, and/or may comprise a genetic construct encoding such a modified RELA protein, wherein the lysine localized at position 310 is substituted for a non-lysine amino acid residue, as compared to the wild type RELA protein of SEQ ID No. 1.

In a particular embodiment, the immune cell according to the invention may comprise and/or express a modified RELA protein derived or issued from a wild-type RELA protein, in particular a wild-type human RELA protein, more particularly a wild-type human RELA protein of SEQ ID No. 1, and/or may comprise a genetic construct encoding such a modified RELA protein, the modified RELA protein having the amino acid sequence set forth in SEQ ID No. 3.

In an aspect of the invention, an immune cell according to the invention may comprise and/or express a modified RELA protein derived or issued from a wild-type RELA protein, in particular a wild-type human RELA protein, more particularly a wild-type human RELA protein of SEQ ID No. 1, and/or may comprise a genetic construct encoding such a modified RELA protein, wherein the lysine localized at position 314 is substituted for a non-lysine amino acid residue, as compared to the wild type RELA protein of SEQ ID No. 1.

In an aspect of the invention, an immune cell according to the invention may comprise and/or express a modified RELA protein derived or issued from a wild-type RELA protein, in particular a wild-type human RELA protein, more particularly a wild-type human RELA protein of SEQ ID No. 1, and/or may comprise a genetic construct encoding such a modified RELA protein, wherein the lysine localized at position 315 is substituted for a non-lysine amino acid residue, as compared to the wild type RELA protein of SEQ ID No. 1.

An immune cell according to the invention may comprise and/or express a modified RELA protein derived or issued from a wild-type RELA protein, in particular a wild-type human RELA protein, more particularly a wild-type human RELA protein of SEQ ID No. 1, and/or may comprise a genetic construct encoding such a modified RELA protein, by substitution of one, two, three, four or five substituted lysine residues as compared to the wild type RELA protein, in particular at positions 122, 123, 310, 314 and/or 315.

In a particular embodiment of the invention, an immune cell according to the invention may comprise and/or express a modified RELA protein derived or issued from a wild-type RELA protein, in particular a wild-type human RELA protein, more particularly a wild-type human RELA protein of SEQ ID No. 1, and/or may comprise a genetic construct encoding such a modified RELA protein, the modified RELA protein exhibiting 5 substituted lysine residues as compared to the wild type RELA protein, in particular at positions 122, 123, 310, 314 and/or 315.

In other words, an immune cell according to the invention may comprise and/or express a modified RELA protein derived or issued from a wild-type RELA protein, in particular a wild-type human RELA protein, more particularly a wild-type human RELA protein of SEQ ID No. 1, and/or may comprise a genetic construct encoding such a modified RELA protein, the modified RELA protein corresponding to a K122 RELA protein, or a K123 RELA protein, or a K310 RELA protein, or a K314 RELA protein, or a K315 RELA protein. The modified RELA protein may correspond to a K122 and K310 RELA protein, or a K123 and K310 RELA protein, or a K310 and K314 RELA protein, or a K310 and K315 RELA protein. An immune cell according to the invention may comprise a modified RELA protein derived or issued from a wild-type RELA protein, in particular a wild-type human RELA protein, more particularly a wild-type human RELA protein of SEQ ID No. 1, and/or may comprise a genetic construct encoding such a modified RELA protein, the modified RELA protein corresponding to a K122 K123 K310 RELA protein, or to a K122 K310 K314 RELA protein, or to a K122 K310 K315 RELA protein, or to a K123 K310 K314 RELA protein. The modified RELA protein may correspond to a K123 K310 K315 RELA protein, or to a K310 K314 K315 RELA protein, or to a K122 K123 K310 K314 RELA protein, or to a K122 K123 K310 K315 RELA protein, or to a K122 K310 K314 K315 RELA protein, or to a K123 K310 K314 K315 RELA protein.

In an embodiment, an immune cell according to the invention may comprise and/or express a modified RELA protein derived or issued from a wild-type RELA protein, in particular a wild-type human RELA protein, more particularly a wild-type human RELA protein of SEQ ID No. 1, and/or may comprise a genetic construct encoding such a modified RELA protein, the modified RELA protein corresponding to a K122 K123 K310 K314 K315 RELA protein. In a particular embodiment, an immune cell according to the invention may comprise and/or express a modified RELA protein derived or issued from a wild-type RELA protein, in particular a wild-type human RELA protein, more particularly a wild-type human RELA protein of SEQ ID No. 1, and/or may comprise a genetic construct encoding such a modified RELA protein, the modified RELA protein having the amino acid sequence set forth in SEQ ID No. 2.

In an aspect of the invention, an immune cell according to the invention may comprise and/or express a modified RELA protein derived or issued from a wild-type RELA protein, in particular a wild-type human RELA protein, more particularly a wild-type human RELA protein of SEQ ID No. 1, and/or may comprise a genetic construct encoding such a modified RELA protein, the modified RELA protein corresponding to the wild type RELA protein, in particular of SEQ ID No. 1, wherein the lysine localized at position 122 is substituted for an arginine residue.

In an aspect of the invention, an immune cell according to the invention may comprise and/or express a modified RELA protein derived or issued from a wild-type RELA protein, in particular a wild-type human RELA protein, more particularly a wild-type human RELA protein of SEQ ID No. 1, and/or may comprise a genetic construct encoding such a modified RELA protein, the modified RELA protein corresponding to the wild type RELA protein, in particular of SEQ ID No. 1, wherein the lysine localized at position 123 is substituted for an arginine residue.

In an aspect of the invention, an immune cell according to the invention may comprise and/or express a modified RELA protein derived or issued from a wild-type RELA protein, in particular a wild-type human RELA protein, more particularly a wild-type human RELA protein of SEQ ID No. 1, and/or may comprise a genetic construct encoding such a modified RELA protein, the modified RELA protein corresponding to the wild type RELA protein, in particular of SEQ ID No. 1, wherein the lysine localized at position 310 is substituted for an arginine residue.

In an aspect of the invention, an immune cell according to the invention may comprise and/or express a modified RELA protein derived or issued from a wild-type RELA protein, in particular a wild-type human RELA protein, more particularly a wild-type human RELA protein of SEQ ID No. 1, and/or may comprise a genetic construct encoding such a modified RELA protein, the modified RELA protein corresponding to the wild type RELA protein, in particular of SEQ ID No. 1, wherein the lysine localized at position 314 is substituted for an arginine residue.

In an aspect of the invention, an immune cell according to the invention may comprise and/or express a modified RELA protein derived or issued from a wild-type RELA protein, in particular a wild-type human RELA protein, more particularly a wild-type human RELA protein of SEQ ID No. 1, and/or may comprise a genetic construct encoding such a modified RELA protein, the modified RELA protein corresponding to the wild type RELA protein, in particular of SEQ ID No. 1, wherein the lysine localized at position 315 is substituted for an arginine residue.

In particular, an immune cell according to the invention may comprise and/or express a modified RELA protein derived or issued from a wild-type RELA protein, in particular a wild-type human RELA protein, more particularly a wild-type human RELA protein of SEQ ID No. 1, and/or may comprise a genetic construct encoding such a modified RELA protein, the modified RELA protein having at least two substituted lysine residues, each being substituted for an arginine residue and are localized at position 122, 123, 310, 314 or 315, more particularly at position 310 and at any other listed position, of the wild-type RELA protein, in particular the wild-type RELA protein of the sequence of SEQ ID No. 1. In a particular embodiment of the invention, an immune cell according to the invention may comprise and/or express a modified RELA protein derived or issued from a wild-type RELA protein, in particular a wild-type human RELA protein, more particularly a wild-type human RELA protein of SEQ ID No. 1, and/or may comprise a genetic construct encoding such a modified RELA protein, the modified RELA protein exhibiting two or more substituted lysine residues substituted for an arginine residue localized i) one at position 310, and ii) one or more at position 122, 123, 314 and/or 315.

In a particular embodiment of the invention, an immune cell according to the invention may comprise and/or express a modified RELA protein derived or issued from a wild-type RELA protein, in particular a wild-type human RELA protein, more particularly a wild-type human RELA protein of SEQ ID No. 1, and/or may comprise a genetic construct encoding such a modified RELA protein, the modified RELA protein exhibiting two, three, four or five substituted lysine residues each substituted for an arginine residue as compared to the wild type RELA protein, in particular at positions 122, 123, 310, 314 and/or 315.

In a particular embodiment of the invention, an immune cell according to the invention may comprise and/or express a modified RELA protein derived or issued from a wild-type RELA protein, in particular a wild-type human RELA protein, more particularly a wild-type human RELA protein of SEQ ID No. 1, and/or may comprise a genetic construct encoding such a modified RELA protein, the modified RELA protein exhibiting 5 substituted lysine residues each substituted for an arginine residue, as compared to the wild type RELA protein, in particular at positions 122, 123, 310, 314 and/or 315.

In other words, an immune cell according to the invention may comprise and/or express a modified RELA protein derived or issued from a wild-type RELA protein, in particular a wild-type human RELA protein, more particularly a wild-type human RELA protein of SEQ ID No. 1, and/or may comprise a genetic construct encoding such a modified RELA protein, the modified RELA protein corresponding to a K122R RELA protein, or a K123R RELA protein, or a K310R RELA protein, or a K314R RELA protein, or a K315R RELA protein, or a K122R and K310R RELA protein, or a K123R and K310R RELA protein, or a K310R and K314R RELA protein, or a K310R and K315R RELA protein, or a K122R K123R K310R RELA protein, or a K122R K310R K314R RELA protein, or a K122R K310R K315R RELA protein, or a K123R K310R K314R RELA protein, or a K123R K310R K315R RELA protein, or a K310R K314R K315R RELA protein, or a K122R K123R K310R K314R RELA protein, or a K122R K123R K310R K315R RELA protein, or a K122R K310R K314R K315R RELA protein or a K123R K310R K314R K315R RELA protein.

In an embodiment an immune cell according to the invention may comprise and/or express a modified RELA protein derived or issued from a wild-type RELA protein, in particular a wild-type human RELA protein, more particularly a wild-type human RELA protein of SEQ ID No. 1, and/or may comprise a genetic construct encoding such a modified RELA protein, the modified RELA protein corresponding to a K122R K123R K310R K314R K315R RELA protein. In a particular embodiment, an immune cell according to the invention may comprise a modified RELA protein derived or issued from a wild-type RELA protein, in particular a wild-type human RELA protein, more particularly a wild-type human RELA protein of SEQ ID No. 1, and/or may comprise a genetic construct encoding such a modified RELA protein, the modified RELA protein having the amino acid sequence set forth in SEQ ID No. 2.

In an embodiment an immune cell according to the invention may comprise and/or express a modified RELA protein having the amino acid sequence set forth in SEQ ID No. 2; or SEQ ID No. 3, or SEQ ID No. 4, or SEQ ID No. 4, or SEQ ID No. 5, or SEQ ID No. 6, or SEQ ID No. 7, or SEQ ID No. 8, or SEQ ID No. 9, or SEQ ID No. 10, or SEQ ID No. 11, or SEQ ID No. 12, or SEQ ID No. 13, or SEQ ID No. 14, or SEQ ID No. 15, or SEQ ID No. 16, or SEQ ID No. 17.

In a particular embodiment, an immune cell according to the invention may comprise and/or express a modified RELA protein derived or issued from a wild-type RELA protein, in particular a wild-type human RELA protein, more particularly a wild-type human RELA protein of SEQ ID No. 1, and/or may comprise a genetic construct encoding such a modified RELA protein, the modified RELA protein is a functional equivalent of human RELA protein and exhibits the modification of the lysine residue(s) herein disclosed. The term “functionally equivalent” includes any equivalent of human RELA protein obtained by altering the amino acid sequence, for example by one or more amino acid deletions, substitutions or additions, in addition to the substitution(s) of lysine residue(s) as disclosed herein, such that the protein analogue retains the ability of wild type RELA protein, in particular its ability to bind to the DNA, in particular to bind to the same localisation within a DNA molecule as compared to a wild type (e.g. unmodified) RELA protein. Amino acid substitutions may be made, for example, by point mutation of the DNA encoding the amino acid sequence.

Immune cells according to the present invention may be cells issued from the lymphoid lineage, including common lymphoid progenitor cells, lymphocytes, natural killer cells, granular lymphocytes, large granular lymphocytes, small lymphocytes, T lymphocytes, and B lymphocytes. In an embodiment of the invention, the immune cells are T cells, in particular any kind of human T cells.

As used herein, the term “T cells” has its general meaning in the art and refers to T lymphocyte which is a type of lymphocyte having a T-cell receptor on the cell surface and playing a central role in cell-mediated immunity. In a particular embodiment, the T cells are human T cells. In a particular embodiment, the T cells are selected from the group consisting of human T cells, CD4+ T cells, CD8+ T cells, naïve T cells, effector T cells, memory T cells, stem cell T cells, central memory T cells, effector memory T cells, terminally differentiated effector memory T cells, tumor-infiltrating lymphocytes, immature T cells, mature T cells, helper T cells, cytotoxic T cells, mucosa-associated invariant T cells, naturally occurring and adaptive regulatory T cells, follicular helper T cells, alpha/beta T cells, CAR-T cells, CD-19 targeting CAR T cells, and delta/gamma T cells. In a preferred embodiment, the T cells are selected from the group consisting of CD4+ T cells, CD8+ T cells, tumor-infiltrating T cells, a genetically engineered T cell expressing chimeric antigen receptors (CARs), and CAR-T cells.

Immune cells according to the invention may be CAR-T cells. T cells may be engineered with CAR molecule. CARs are localized within the membrane of T cells. A CAR is a chimeric molecule comprising as its extracellular part an antibody-derived antigen recognition domain (usually an ScFv fragment), and as its intracellular domain a TCR-derived activating domain which confers to the T cells the capability to be activated against a specific tumor antigen (Gomes-Silva et al., Biotech J. 2017). The clinical results of the murine derived CART 19 (i.e. “CTL019”) have shown some complete remissions in patients suffering from CLL (Chronic lymphocytic leukemia) as well as childhood ALL (Acute lymphocytic leukemia) (Grupp et al., 2013; Kalos et al., 2011; Porter et al., 2011). Novel targets for CAR T cell therapy against solid tumors are currently under development. Such strategy may have high clinical potential. CAR T cells can be engineered for targeting antigens, thereby providing putative broad applications.

The antigen is a tumor antigen, which can be for example selected from the group consisting of CD19, MUC16, MUC1, CA1X, CEA, CD8, CD7, CD10, CD20, CD22, CD30, CLL1, CD33, CD34, CD38, CD41, CD44, CD49f, CD56, CD74, CD133, CD138, EGP-2, EGP-40, EpCAM, erb-B2,3,4, FBP, Fetal acetylcholine receptor, folate receptor-a, GD2, GD3, ITER-2, hTERT, IL-13R-a2, K-light chain, KDR, LeY, LI cell adhesion molecule, MAGE-A1, Mesothelin, ERBB2, MAGEA3, p53, MARTI, GPI00, Proteinase3 (PR1), Tyrosinase, Survivin, hTERT, EphA2, NKG2D ligands, NY-ESO-1, oncofetal antigen (h5T4), PSCA, PSMA, ROR1, TAG-72, VEGF-R2, WT-I, BCMA, CD123, CD44V6, NKCS1, HER2, EGF1R, EGFR-VIII, and CD99, CD70, ADGRE2, CCR1, LILRB2, PRAME, CCR4, CD5, CD3, TRBC1, TRBC2, TIM-3, Integrin B7, ICAM-1, CD70, Tim3, CLEC12A and ER.

Among the antigens targeted by the antigen-specific receptors of the CAR T cells are those expressed in the context of a disease, condition, or cell type to be targeted via the adoptive cell therapy. Among the diseases and conditions are proliferative, neoplastic, and malignant diseases and disorders, more particularly cancers. Infectious diseases and autoimmune, inflammatory or allergic diseases are also contemplated.

The cancer may be a “solid cancer” or a “liquid tumor” such as cancers affecting the blood, bone marrow and lymphoid system, also known as tumors of the hematopoietic and lymphoid tissues, which notably include leukemia and lymphoma. Liquid tumors include for example acute myelogenous leukemia (AML), chronic myelogenous leukemia (CML), acute lymphocytic leukemia (ALL), and chronic lymphocytic leukemia (CLL), (including various lymphomas such as mantle cell lymphoma, non-Hodgkins lymphoma (NHL), adenoma, squamous cell carcinoma, laryngeal carcinoma, gallbladder and bile duct cancers, cancers of the retina such as retinoblastoma).

Solid cancers notably include cancers affecting one of the organs selected from the group consisting of colon, rectum, skin, endometrium, lung (including non-small cell lung carcinoma), uterus, bones (such as Osteosarcoma, Chondrosarcomas, Ewing's sarcoma, Fibrosarcomas, Giant cell tumors, Adamantinomas, and Chordomas), liver, kidney, esophagus, stomach, bladder, pancreas, cervix, brain (such as Meningiomas, Glioblastomas, Lower-Grade Astrocytomas, Oligodendrocytomas, Pituitary Tumors, Schwannomas, and Metastatic brain cancers), ovary, breast, head and neck region, testis, prostate and the thyroid gland.

Preferably, a cancer according to the invention is a cancer affecting the blood, bone marrow and lymphoid system as described above. In some embodiments, the cancer is, or is associated, with multiple myeloma.

Diseases according to the invention also encompass infectious diseases or conditions, such as, but not limited to, viral, retroviral, bacterial, and protozoal infections, HIV immunodeficiency, Cytomegalovirus (CMV), Epstein-Barr virus (EBV), adenovirus, BK polyomavirus.

Diseases according to the invention also encompass autoimmune or inflammatory diseases or conditions, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease multiple sclerosis, asthma, and/or diseases or conditions associated with transplant.

In some embodiments, the antigen is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells. In some such embodiments, a multi-targeting and/or gene disruption approach as provided herein is used to improve specificity and/or efficacy.

In some embodiments, the antigen is a universal tumor antigen. The term “universal tumor antigen” refers to an immunogenic molecule, such as a protein, that is, generally, expressed at a higher level in tumor cells than in non-tumor cells and also is expressed in tumors of different origins. In some embodiments, the universal tumor antigen is expressed in more than 30%, 40%, 50%, 60%, 70%, 75%, 80%, 85%, 90% or more of human cancers. In some embodiments, the universal tumor antigen is expressed in at least three, at least four, at least five, at least six, at least seven, at least eight or more different types of tumors.

In some cases, the universal tumor antigen may be expressed in non-tumor cells, such as normal cells, but at lower levels than it is expressed in tumor cells. In some cases, the universal tumor antigen is not expressed at all in non-tumor cells, such as not expressed in normal cells. Exemplary universal tumor antigens include, for example, human telomerase reverse transcriptase (hTERT), survivin, mouse double minute 2 homolog (MDM2), cytochrome P450 1B1 (CYP1B), HER2/neu, p95HER2, Wilms' tumor gene 1 (WT1), livin, alphafetoprotein (AFP), carcinoembryonic antigen (CEA), mucin 16 (MUC16), MUC1, prostate-specific membrane antigen (PSMA), p53 or cyclin (DI). Peptide epitopes of tumor antigens, including universal tumor antigens, are known in the art and, in some aspects, can be used to generate MHC-restricted antigen-specific receptors, such as TCRs or TCR-like CARs (see e.g. published PCT application No. WO2011009173 or WO2012135854 and published U.S. application No. US20140065708).

In some aspects, the antigen is expressed on multiple myeloma, such as CD38, CD138, and/or CS-1. Other exemplary multiple myeloma antigens include CD56, TIM-3, CD33, CD123, and/or CD44. Antibodies or antigen-binding fragments directed against such antigens are known and include, for example, those described in U.S. Pat. Nos. 8,153,765; 8,603,477, 8,008,450; U.S. published application No. US20120189622; and published international PCT application Nos. WO2006099875, WO2009080829 or WO2012092612. In some embodiments, such antibodies or antigen-binding fragments thereof (e.g. scFv) can be used to generate a CAR.

In some embodiments, the antigen may be one that is expressed or upregulated on cancer or tumor cells, but that also may be expressed in an immune cell, such as a resting or activated T cell. For example, in some cases, expression of hTERT, survivin and other universal tumor antigens are reported to be present in lymphocytes, including activated T lymphocytes (see e.g., Weng et al. (1996) J Exp. Med., 183:2471-2479; Hathcock et al. (1998) J Immunol, 160:5702-5706; Liu et al. (1999) Proc. Natl Acad Sci., 96:5147-5152; Turksma et al. (2013) Journal of Translational Medicine, 11: 152).

In some embodiments, the cancer is, or is associated, with overexpression of HER2 or p95HER2. p95HER2 is a constitutively active C-terminal fragment of HER2 that is produced by an alternative initiation of translation at methionine 611 of the transcript encoding the full-length HER2 receptor. HER2 or p95HER2 has been reported to be overexpressed in breast cancer, as well as gastric (stomach) cancer, gastroesophageal cancer, esophageal cancer, ovarian cancer, uterine endometrial cancer, cervix cancer, colon cancer, bladder cancer, lung cancer, and head and neck cancers. Patients with cancers that express the p95HER2 fragment have a greater probability of developing metastasis and a worse prognosis than those patients who mainly express the complete form of HER2. Saez et al., Clinical Cancer Research, 12:424-431 (2006).

In some embodiments as provided herein, an immune cell, such as a T cell, can be engineered to repress or disrupt the gene encoding the antigen in the immune cell so that the expressed antigen-specific receptor does not specifically bind the antigen in the context of its expression on the immune cell itself. Thus, in some aspects, this may avoid off-target effects, such as binding of the engineered immune cells to themselves, which may reduce the efficacy of the engineered in the immune cells, for example, in connection with adoptive cell therapy.

In some embodiments, such as in the case of an inhibitory CAR, the target is an off-target marker, such as an antigen not expressed on the diseased cell or cell to be targeted, but that is expressed on a normal or non-diseased cell which also expresses a disease-specific target being targeted by an activating or stimulatory receptor in the same engineered cell. Exemplary such antigens are MHC molecules, such as MHC class I molecules, for example, in connection with treating diseases or conditions in which such molecules become downregulated but remain expressed in non-targeted cells.

In some embodiments, the engineered immune cells can contain an antigen-specific receptor that targets one or more other antigens. In some embodiments, the one or more other antigens is a tumor antigen or cancer marker. Other antigen targeted by antigen-specific receptors on the provided immune cells can, in some embodiments, include orphan tyrosine kinase receptor ROR1, tEGFR, Her2, p95HER2, LI-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, EPHa2, ErbB2, 3, or 4, FBP, fetal acethycholine e receptor, GD2, GD3, HMW-MAA, IL-22R-alpha, IL-13R-alpha2, kdr, kappa light chain, Lewis Y, LI-cell adhesion molecule, MAGE-A1, mesothelin, MUC1, MUC16, PSCA, NKG2D Ligands, NY-ESO-1, MART-1, gplOO, oncofetal antigen, ROR1, TAG72, VEGF-R2, carcinoembryonic antigen (CEA), prostate specific antigen, PSMA, Her2/neu, p95HER2, estrogen receptor, progesterone receptor, ephrinB2, CD123, CS-1, c-Met, GD-2, and MAGE A3, CE7, Wilms Tumor 1 (WT-1), a cyclin, such as cyclin AI (CCNA1), and/or biotinylated molecules, and/or molecules expressed by HIV, HCV, HBV or other pathogens, such as gp120 (but see also Kuhlmann A S, Peterson C W, Kiem H P. Chimeric antigen receptor T-cell approaches to HIV cure. Curr Opin HIV AIDS. 2018 September; 13(5):446-453).

In some embodiments, the CAR binds a pathogen-specific antigen. In some embodiments, the CAR is specific for viral antigens (such as HIV, HCV, HBV, etc.), bacterial antigens, and/or parasitic antigens.

In some embodiments, the cell of the invention is genetically engineered to express two or more antigen-specific receptors on the cell, each recognizing a different antigen and typically each including a different intracellular signaling component. Such multi-targeting strategies are described, for example, in International Patent Application, Publication No.: WO 2014055668 A1 (describing combinations of activating and costimulatory CARs, e.g., targeting two different antigens present individually on off-target, e.g., normal cells, but present together only on cells of the disease or condition to be treated) and Fedorov et al., Sci. Transl. Medicine, 5(215) (December, 2013) (describing cells expressing an activating and an inhibitory CAR, such as those in which the activating CAR binds to one antigen expressed on both normal or non-diseased cells and cells of the disease or condition to be treated, and the inhibitory CAR binds to another antigen expressed only on the normal cells or cells which it is not desired to treat). Example antigen-binding receptors include bispecific antibodies that are T-cell activating antibodies which bind not only the desired antigen but also an activating T-cell antigen such as CD3 epsilon.

In some contexts, overexpression of a stimulatory factor (for example, a lymphokine or a cytokine) may be toxic to a subject. Thus, in some contexts, the engineered cells include gene segments that cause the cells to be susceptible to negative selection in vivo, such as upon administration in adoptive cell therapy.

For example in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-1 TK) gene (Wigler et al., Cell II: 223, 1977) which confers ganciclovir sensitivity; the cellular hypoxanthine phosphribosyltransferase (HPRT) gene, the cellular adenine phosphoribosyltransferase (APRT) gene, bacterial cytosine deaminase, (Mullen et al., Proc. Natl. Acad. Sci. USA. 89:33 (1992)). In some embodiments, the engineered immune cells can contain an antigen-specific receptor that targets one or more other antigens. In some embodiments, the one or more other antigens is a tumor antigen or cancer marker. Other antigen targeted by antigen-specific receptors on the provided immune cells can, in some embodiments, include orphan tyrosine kinase receptor ROR1, tEGFR, Her2, p95HER2, LI-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, EPHa2, ErbB2, 3, or 4, FBP, fetal acetylcholine e receptor, GD2, GD3, HMW-MAA, IL-22R-alpha, IL-13R-alpha2, kdr, kappa light chain, Lewis Y, LI-cell adhesion molecule, MAGE-A1, mesothelin, MUC1, MUC16, PSCA, NKG2D Ligands, NY-ESO-1, MART-1, gplOO, oncofetal antigen, ROR1, TAG72, VEGF-R2, carcinoembryonic antigen (CEA), prostate specific antigen, PSMA, Her2/neu, p95HER2, estrogen receptor, progesterone receptor, ephrinB2, CD 123, CS-1, c-Met, GD-2, and MAGE A3, CE7, Wilms Tumor 1 (WT-1), a cyclin, such as cyclin AI (CCNA1), and/or biotinylated molecules, and/or molecules expressed by HIV, HCV, HBV or other pathogens.

In some embodiments, the CAR binds a pathogen-specific antigen. In some embodiments, the CAR is specific for viral antigens (such as HIV, HCV, HBV, etc.), bacterial antigens, and/or parasitic antigens.

In some embodiments, the cells of the invention is genetically engineered to express two or more antigen-specific receptors on the cell, each recognizing a different antigen and typically each including a different intracellular signaling component. Such multi-targeting strategies are described, for example, in International Patent Application, Publication No.: WO 2014055668 A1 (describing combinations of activating and costimulatory CARs, e.g., targeting two different antigens present individually on off-target, e.g., normal cells, but present together only on cells of the disease or condition to be treated) and Fedorov et al., Sci. Transl. Medicine, 5(215) (December, 2013) (describing cells expressing an activating and an inhibitory CAR, such as those in which the activating CAR binds to one antigen expressed on both normal or non-diseased cells and cells of the disease or condition to be treated, and the inhibitory CAR binds to another antigen expressed only on the normal cells or cells which it is not desired to treat).

In some contexts, the engineered cells include gene segments that cause the cells to be susceptible to negative selection in vivo, such as upon administration in adoptive cell therapy. For example in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-1 TK) gene (Wigler et al., Cell II: 223, 1977) which confers ganciclovir sensitivity; the cellular hypoxanthine phosphribosyltransferase (HPRT) gene, the cellular adenine phosphoribosyltransferase (APRT) gene, bacterial cytosine deaminase, (Mullen et al., Proc. Natl. Acad. Sci. USA. 89:33 (1992)).

In other embodiments of the invention, the cells, i.e., myeloid cells (typically dendritic cells or phagocytic cells such as macrophages), are not engineered to express recombinant antigen-specific receptors, but rather include naturally occurring antigen-specific receptors specific for desired antigens, such dendritic cells, monocytes, macrophages or their progenitors cultured in vitro or ex vivo, e.g., during the incubation step(s), to promote expansion of cells having particular antigen specificity.

In a particular embodiment, the immune cell is isolated.

In a particular embodiment, the immune cell is a human cell.

In a particular embodiment, the immune cell is a cell line or is issued from a cell line.

According to an embodiment of the invention, the immune cells are further modified to overexpress IRF3 protein (interferon Regulatory Factor 3). Overexpression of IRF3 in an immune cell of the invention may be assessed by comparison with the expression of IRF3 in wild type (i.e. unmodified cell) of the invention.

In an aspect of the invention, the immune cell of the invention is used for treating a NF-κB-associated disease. Aberrant NF-κB activation contributes to development of various autoimmune, inflammatory, and malignant disorders including rheumatoid arthritis, atherosclerosis, inflammatory bowel diseases, multiple sclerosis and malignant tumors. NF—KB is able to induce several cellular alterations and has been shown to be constitutively activated in some types of cancer cells. In an aspect of the invention, the modified immune cells, in particular modified T cells, more particularly CAR-T cells, are used for treating patient having multiple sclerosis.

In an aspect of the invention, the immune cell of the invention is used for treating an infection, more particularly a viral infection. In a more particular aspect, the modified RELA protein is used for treating a viral infection caused by a retrovirus or a lentivirus. In a more particular aspect, the modified RELA protein is used for treating an infection by a HIV, in particular HIV-I or HIV-II.

In an aspect of the invention, the immune cell of the invention is used for treating a patient infected by a virus, in particular infected by a retrovirus or a lentivirus, more particularly infected by a HIV, like HIV-I or HIV-II.

In an aspect of the invention, the immune cell of the invention is used for treating a patient having a cancer, in particular a patient having a bladder cancer, bone cancer, brain cancer, breast cancer, cervical cancer, colon cancer, esophageal cancer, gastric cancer, head & neck cancers, hodgkin's lymphoma, leukemia, liver cancer, lung cancer, melanoma, mesothelioma, multiple myeloma, myelodysplastic syndrome, non-hodgkin's lymphoma, ovarian cancer, pancreatic cancer, prostate cancer, rectal cancer, renal cancer, sarcoma, skin cancer, testicular cancer, thyroid cancer or uterine cancer. In a particular embodiment, the cancer which affect a patient is a lung cancer, more particularly a lung carcinoma.

### Modified RELA Protein

According to a second aspect, it is provided a modified RELA protein for modifying the metabolism of interferon in immune cells, in particular T cells. In an aspect, it is provided a modified RELA protein, in particular a modified human RELA protein, wherein the modified RELA protein is derived from a wild-type RELA protein, in particular a wild-type human RELA protein, more particularly a wild-type human RELA protein of SEQ ID No. 1, by substitution of at least one lysine (K) residue for a non-lysine residue, for use in the elicitation of the production of interferon (IFN), in particular IFN-1 and/or IFN-III, in immune cells, in particular in T cells.

RELA protein is also known under as the transcription factor p65 or nuclear factor NF-kappa-B p65 subunit. These three terms are used interchangeably within the whole description of the present invention. RELA protein is a protein that in humans is encoded by the RELA gene. RELA protein is a REL-Associated protein involved in NF-κB heterodimer formation, and its nuclear translocation and activation. Phosphorylation and acetylation of RELA are crucial post-translational modifications required for NF-κB activation. NF-kappa-B is a homo- or heterodimeric complex formed by the Rel-like domain-containing proteins RELA/p65, RELB, NFKB1/p105, NFKB1/p50, REL and NFKB2/p52. The heterodimeric RELA-NFKB1 complex is one of the most abundant form of NF-κB. The wild type RELA protein may correspond to the Uniprot reference Q04206. The wild type RELA protein may have the amino acid sequence of SEQ ID No. 1.

In an embodiment, the modified RELA protein can bind to DNA implicated in IFN-I expression, like a wild type RELA protein. The ability to bind to DNA implicated in IFN-I expression may be assessed by methods known by the skilled artisan, for example by competition binding between a modified RELA protein and a wild type RELA protein on DNA implicated in the IFN-I expression.

In an embodiment, the modified RELA protein induces IFN-I production in immune cells, in particular in T cells.

In an embodiment, the modified RELA protein can bind to DNA implicated in IFN-I expression, like a wild type RELA protein, and the modified RELA protein induces IFN-I production in immune cells, in particular in T cells.

In an embodiment, the lysine residue is substituted for an amino acid residue that cannot be acetylated.

By derived from a wild-type RELA protein, it should be understood that the modified RELA protein of the invention is modified as compared to the wild-type RELA protein from which it is derived by mutation, including substitution (including conservative amino acid residue(s)) and/or by addition and/or deletion of amino acid residue(s) and/or by secondary modification after translation and/or by deletion of portion(s) of the wild-type RELA protein (resulting in a modified RELA protein having a shortened size with respect to the wild-type RELA protein of reference). Fragments of the RELA protein are encompassed within the present invention to the extent that they possess the same functional properties, in particular DNA binding on particular localization, as compared to a wild-type RELA protein.

In the present description, the expression “modified RELA protein” corresponds to a RELA protein with substituted lysine residue(s) as defined herein as compared to a wild type RELA protein. Wild type RELA protein may partially, or fully, correspond to the amino acid sequence set forth in SEQ ID No: 1 (human RELA protein). A modified RELA protein may correspond to a protein having at least 70%, preferably at least 80%, more preferably at least 90%, and most preferably at least 95% of identity with the amino acid sequence of the wild type RELA protein defined herein, the modified RELA protein further exhibiting the substitution of at least one lysine residue of the wild type RELA protein as defined herein. As compared to a wild type RELA protein, e.g. a RELA protein naturally found in human being, the modified RELA protein of the invention is mutated as compared to the wild type RELA protein by at least the substitution of at least one lysine residue for a non-lysine residue. In other words, the RELA protein may exhibit other mutations than the one required according to the invention. The modified RELA protein may be a modified human protein, a recombinant (and human) RELA protein. By non-lysine residue, it means that any other amino acid residue than lysine can be present within the modified RELA protein in replacement of the lysine residue presents in the wild type version of the RELA protein. In a particular embodiment, functional equivalent of RELA protein exhibits at least 70%, preferably at least 80%, more preferably at least 90%, and most preferably at least 95% of identity with the amino acid sequence of the wild type RELA protein defined herein.

In an aspect of the invention, the modified RELA protein shares at least 90% identity with the wild-type RELA protein of SEQ ID No. 1, but has a lysine localized at position 122 of SEQ ID No. 1. In an aspect of the invention, the modified RELA protein shares at least 90% identity with the wild-type RELA protein of SEQ ID No. 1, but has a lysine localized at position 123 of SEQ ID No. 1. In an aspect of the invention, the modified RELA protein shares at least 90% identity with the wild-type RELA protein of SEQ ID No. 1, but has a lysine localized at position 310 of SEQ ID No. 1. In an aspect of the invention, the modified RELA protein shares at least 90% identity with the wild-type RELA protein of SEQ ID No. 1, but has a lysine localized at position 314 of SEQ ID No. 1. In an aspect of the invention, the modified RELA protein shares at least 90% identity with the wild-type RELA protein of SEQ ID No. 1, but has a lysine localized at position 315 of SEQ ID No. 1. In an aspect of the invention, the modified RELA protein shares at least 90% identity with the wild-type RELA protein of SEQ ID No. 1, but has lysine localized at positions 122, 123, 310, 314 and 315 of SEQ ID No. 1.

In an aspect of the invention, the modified RELA protein corresponds to the wild type RELA protein, in particular of SEQ ID No. 1, wherein the lysine localized at position 122 is substituted for a non-lysine amino acid residue.

In an aspect of the invention, the modified RELA protein corresponds to the wild type RELA protein, in particular of SEQ ID No. 1, wherein the lysine localized at position 123 is substituted for a non-lysine amino acid residue.

In an aspect of the invention, the modified RELA protein corresponds to the wild type RELA protein, in particular of SEQ ID No. 1, wherein the lysine localized at position 310 is substituted for a non-lysine amino acid residue. In a particular embodiment, the modified RELA protein may have the amino acid sequence set forth in SEQ ID No. 3.

In an aspect of the invention, the modified RELA protein corresponds to the wild type RELA protein, in particular of SEQ ID No. 1, wherein the lysine localized at position 314 is substituted for a non-lysine amino acid residue.

In an aspect of the invention, the modified RELA protein corresponds to the wild type RELA protein, in particular of SEQ ID No. 1, wherein the lysine localized at position 315 is substituted for a non-lysine amino acid residue.

In a particular embodiment of the invention, the modified RELA protein exhibits more than one substituted lysine residue as compared to the wild type RELA protein. In particular, at least two substituted lysine residues are localized at position 122, 123, 310, 314 or 315, more particularly at position 310 and at any other listed position, of the wild-type RELA protein, in particular the wild-type RELA protein of the sequence of SEQ ID No. 1. In a particular embodiment of the invention, the modified RELA protein exhibits two or more substituted lysine residues localized i) one at position 310, and ii) one or more at position 122, 123, 314 and/or 315.

In a particular embodiment of the invention, the modified RELA protein exhibits two, three, four or five substituted lysine residues as compared to the wild type RELA protein, in particular at positions 122, 123, 310, 314 and/or 315.

In a particular embodiment of the invention, the modified RELA protein exhibits 5 substituted lysine residues as compared to the wild type RELA protein, in particular at positions 122, 123, 310, 314 and/or 315.

In other words, the modified RELA protein may correspond to a K122 RELA protein, or a K123 RELA protein, or a K310 RELA protein, or a K314 RELA protein, or a K315 RELA protein. The modified RELA protein may correspond to a K122 and K310 RELA protein, or a K123 and K310 RELA protein, or a K310 and K314 RELA protein, or a K310 and K315 RELA protein. The modified RELA protein may correspond to a K122 K123 K310 RELA protein. The modified RELA protein may correspond to a K122 K310 K314 RELA protein. The modified RELA protein may correspond to a K122 K310 K315 RELA protein. The modified RELA protein may correspond to a K123 K310 K314 RELA protein. The modified RELA protein may correspond to a K123 K310 K315 RELA protein. The modified RELA protein may correspond to a K310 K314 K315 RELA protein. The modified RELA protein may correspond to a K122 K123 K310 K314 RELA protein. The modified RELA protein may correspond to a K122 K123 K310 K315 RELA protein. The modified RELA protein may correspond to a K122 K310 K314 K315 RELA protein. The modified RELA protein may correspond to a K123 K310 K314 K315 RELA protein.

In an embodiment the modified RELA protein may correspond to a K122 K123 K310 K314 K315 RELA protein.

In an embodiment of the invention, at least one substituted lysine residue is substituted for an arginine (R) residue. In an embodiment, each substituted lysine residue is substituted for an arginine (R) residue.

In an aspect of the invention, the modified RELA protein corresponds to the wild type RELA protein, in particular of SEQ ID No. 1, wherein the lysine localized at position 122 is substituted for an arginine residue.

In an aspect of the invention, the modified RELA protein corresponds to the wild type RELA protein, in particular of SEQ ID No. 1, wherein the lysine localized at position 123 is substituted for an arginine residue.

In an aspect of the invention, the modified RELA protein corresponds to the wild type RELA protein, in particular of SEQ ID No. 1, wherein the lysine localized at position 310 is substituted for an arginine residue.

In an aspect of the invention, the modified RELA protein corresponds to the wild type RELA protein, in particular of SEQ ID No. 1, wherein the lysine localized at position 314 is substituted for an arginine residue.

In an aspect of the invention, the modified RELA protein corresponds to the wild type RELA protein, in particular of SEQ ID No. 1, wherein the lysine localized at position 315 is substituted for an arginine residue.

In particular, at least two substituted lysine residues are each substituted for an arginine residue and are localized at position 122, 123, 310, 314 or 315, more particularly at position 310 and at any other listed position, of the wild-type RELA protein, in particular the wild-type RELA protein of the sequence of SEQ ID No. 1. In a particular embodiment of the invention, the modified RELA protein exhibits two or more substituted lysine residues substituted for an arginine residue localized i) one at position 310, and ii) one or more at position 122, 123, 314 and/or 315.

In a particular embodiment of the invention, the modified RELA protein exhibits more two, three, four or five substituted lysine residues each substituted for an arginine residue as compared to the wild type RELA protein, in particular at positions 122, 123, 310, 314 and/or 315.

In a particular embodiment of the invention, the modified RELA protein exhibits 5 substituted lysine residues each substituted for an arginine residue, as compared to the wild type RELA protein, in particular at positions 122, 123, 310, 314 and/or 315.

In other words, the modified RELA protein may correspond to a K122R RELA protein, or a K123R RELA protein, or a K310R RELA protein, or a K314R RELA protein, or a K315R RELA protein. The modified RELA protein may correspond to a K122R and K310R RELA protein, or a K123R and K310R RELA protein, or a K310R and K314R RELA protein, or a K310R and K315R RELA protein. The modified RELA protein may correspond to a K122R K123R K310R RELA protein.

The modified RELA protein may correspond to a K122R K310R K314R RELA protein. The modified RELA protein may correspond to a K122R K310R K315R RELA protein. The modified RELA protein may correspond to a K123R K310R K314R RELA protein. The modified RELA protein may correspond to a K123R K310R K315R RELA protein. The modified RELA protein may correspond to a K310R K314R K315R RELA protein. The modified RELA protein may correspond to a K122R K123R K310R K314R RELA protein. The modified RELA protein may correspond to a K122R K123R K310R K315R RELA protein. The modified RELA protein may correspond to a K122R K310R K314R K315R RELA protein. The modified RELA protein may correspond to a K123R K310R K314R K315R RELA protein.

In an embodiment of the invention, a modified RELA protein has the amino acid sequence set forth in SEQ ID No. 2; or SEQ ID No. 3, or SEQ ID No. 4, or SEQ ID No. 4, or SEQ ID No. 5, or SEQ ID No. 6, or SEQ ID No. 7, or SEQ ID No. 8, or SEQ ID No. 9, or SEQ ID No. 10, or SEQ ID No. 11, or SEQ ID No. 12, or SEQ ID No. 13, or SEQ ID No. 14, or SEQ ID No. 15, or SEQ ID No. 16, or SEQ ID No. 17.

In an embodiment the modified RELA protein may correspond to a K122R K123R K310R K314R K315R RELA protein. In a particular embodiment, the modified RELA protein may have the amino acid sequence set forth in SEQ ID No. 2.

In an embodiment, the modified RELA protein is a functional equivalent of human RELA protein and exhibits the modification of the lysine residue(s) herein disclosed. The term “functionally equivalent” thus includes any equivalent of human RELA protein obtained by altering the amino acid sequence, for example by one or more amino acid deletions, substitutions or additions, in addition to the substitution(s) of lysine residue(s) as disclosed herein, such that the protein analogue retains the ability of wild type RELA protein, in particular its ability to bind to the DNA, in particular to bind to the same localisation within a DNA molecule as compared to a wild type RELA protein. Amino acid substitutions may be made, for example, by point mutation of the DNA encoding the amino acid sequence.

Any modified RELA protein disclosed herein may be for use in the elicitation or the enhancement of the production of interferon (IFN), in particular IFN-1 and/or IFN-III, more particularly IFN-α, IFN-β, IFN-λ and/or IFN-II, in immune cells, in particular in T cells. In a particular embodiment of the invention, the use is for the elicitation or the enhancement of the production of IFN-I by T-cells.

The administration of the modified RELA protein of the invention. to immune cells, or the expression of the modified RELA protein of the invention. by immune cells leads to an improved production of interferon by the immune cells. The production of the interferon by the immune cells may be assessed by comparison with a negative control, e.g. an immune cell of the same type that does not express the modified RELA protein and that is not in contact with a modified RELA protein of the invention. The measurement of interferon production may be assessed according to the method disclose din the working example of the invention, in particular the material and method associated with FIG. 1F of the examples.

Any modified RELA protein disclosed herein may be for use in the elicitation or the enhancement of IRF7 expression in immune cells, in particular in T cells.

Any modified RELA protein disclosed herein may be for use in the elicitation or the enhancement of IFN production, in particular IFN-I or IFN-III, more particularly IFN-I, in response to STING stimulation. STING stimulation may correspond to the method disclosed in the wording examples of the invention.

In an aspect of the invention, the modified RELA protein is used for promoting interferon production by immune cells, in particular by T cells.

In an aspect of the invention, the modified RELA protein is used for providing immune cells, in particular T cells, with improved anti-tumor activity, as compared to a negative control. Anti-tumor activity may be assessed according to the working example of the invention, and an improvement in the anti-tumor activity of immune cells may be assessed by comparison with a negative control. A negative control may consist in immune cells of the same type, but that do not express and that are not in contact with the modified RELA protein of the invention.

In an aspect of the invention, the modified RELA protein is used for providing immune cells, in particular T cells, with improved anti-infection resistance. Anti-infection resistance may be assessed according to the working example of the invention, and an improvement in the anti-infection resistance of immune cells may be assessed by comparison with a negative control. A negative control may consist in immune cells of the same type, but that do not express and that are not in contact with the modified RELA protein of the invention.

In an aspect of the invention, the modified RELA protein is used for enhancing the T cell response against a disease or a pathogen. As used herein, the term “T cells response” refers to any biological process involving T cells proliferation and/or cytokine synthesis. The T cells response can be determined by various methods well known from one skilled in the art by assessing T cells proliferation and/or cytokine synthesis. In one embodiment, T cells response is determined by measuring the IFN expression. IFN expression can be assessed according to any method disclosed in the examples of the invention.

In an aspect of the invention, the modified RELA protein is used for treating a NF-κB-associated disease. Aberrant NF-κB activation contributes to development of various autoimmune, inflammatory, and malignant disorders including rheumatoid arthritis, atherosclerosis, inflammatory bowel diseases, multiple sclerosis and malignant tumors. NF—KB is able to induce several cellular alterations and has been shown to be constitutively activated in some types of cancer cells.

In an aspect of the invention, the modified RELA protein is used for treating an infection, more particularly a viral infection. In a more particular aspect, the modified RELA protein is used for treating a viral infection caused by a retrovirus or a lentivirus. In a more particular aspect, the modified RELA protein is used for treating an infection by a HIV, in particular HIV-I or HIV-II.

In an aspect of the invention, the modified RELA protein is used for treating a patient infected by a virus, in particular infected by a retrovirus or a lentivirus, more particularly infected by a HIV, like HIV-I or HIV-II.

In an aspect of the invention, the modified RELA protein is used for treating a patient having a cancer, in particular a patient having a bladder cancer, bone cancer, brain cancer, breast cancer, cervical cancer, colon cancer, oesophageal cancer, gastric cancer, head & neck cancers, Hodgkin's lymphoma, leukaemia, liver cancer, lung cancer, melanoma, mesothelioma, multiple myeloma, myelodysplastic syndrome, non-Hodgkin's lymphoma, ovarian cancer, pancreatic cancer, prostate cancer, rectal cancer, renal cancer, sarcoma, skin cancer, testicular cancer, thyroid cancer or uterine cancer. In a particular embodiment, the cancer which affect a patient is a lung cancer, more particularly a lung carcinoma.

### Genetic Construct Encoding a Modified RELA Protein

In another aspect of the invention, it is provided a genetic construct or a gene transfer vector encoding a modified RELA protein according to any embodiment disclosed herein, for use in the engineering of immune cells, in particular T cells. Immune cells engineered accordingly exhibit an improved production of interferon, in particular IFN-I and/or IFN-III.

The genetic construct or gene transfer vector may be a nucleic acid molecule encoding at least the amino acid sequence corresponding to at least one modified RELA protein as disclosed herein.

The genetic construct or gene transfer vector may be a polynucleotide encoding at least the amino acid sequence of SEQ DI No. 2 or SEQ ID No. 3.

The genetic construct or gene transfer vector may be a vector for the cloning and/or for the expression of a nucleic acid molecule encoding at least the amino acid sequence corresponding to any modified RELA protein as disclosed herein.

In particular, said vector is a plasmid suitable for cloning and/or expressing in mammalian cells, which comprises regulation sequences for transcription and expression. Accordingly, provided herein is a vector comprising a polynucleotide sequence encoding at least one modified RELA protein as disclosed herein In a particular embodiment, the genetic construct or gene transfer vector is isolated.

The term “genetic construct” or “gene transfer vector” may refer to a vector suitable for expression of a gene in a cell, or a viral vector comprising in its genome a vector plasmid, a vector DNA, a polynucleotide construct or nucleic acid construct or nucleic material issued from virus (i.e. derived from the genome of a virus), wherein the vector is transferred into a cell, a cell line or a host cell, thereby allowing the transcription of a heterologous polynucleotide encoding the modified RELA protein inserted within its sequence or genome into mRNA and its further translation into a functional protein, in particular within a mammalian cell, in particular within a human cell. The term “gene transfer vector” may refer to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “gene transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like. A gene transfer vector may be derived from a virus (i.e. virus-based vector), such as a viral vector, also designated a viral vector particle or a viral-gene transfer vector. Alternatively, the gene transfer vector may be a non-viral gene transfer vector, such as a plasmid, and designated for the expression of the heterologous polynucleotide encoding the modified RELA protein and present within the gene transfer vector, in order to produce within immune cells, in particular T cell(s), at least the modified RELA protein encoded by the heterologous polynucleotide. In other words, the gene transfer vector, once inserted within immune cells, in particular T cell(s), allows the production of significant amount of mRNA, which are then translated into protein within the cytoplasm of modified (or engineered) immune cells, in particular T cell(s). As non-limitative examples, the following gene transfer vectors may be used in a method according to the invention: a plasmid, a viral vector, an artificial chromosome, in particular human artificial chromosomes. In a particular embodiment of the invention, the gene transfer vector is a mammalian gene transfer vector, i.e. allowing the transcription and the further translation of the heterologous polynucleotide in a mammalian immune cell in particular a T cell, in particular in a human T cell. In a particular embodiment of the invention, the gene transfer vector is issued or derived from a virus, in particular it is produced or rescued from the genome of a modified virus in a manner which is known to a person skilled in the art, such as by deletion of viral nucleotide sequences involved in expression of structural proteins of the virus. A gene transfer vector may therefore be a viral vector based or issued from an adenovirus or an adeno-associated virus. More particularly, the gene transfer vector may be based or issued or derived from a retrovirus, in particular an alpha-retrovirus, a beta-retrovirus, a gamma-retrovirus, delta-retrovirus, an endogenous retrovirus, or a lentivirus. More particularly, the gene transfer vector is a retroviral vector or a lentiviral vector. The gene transfer vector may furthermore comprise enhancer(s), inducer(s) and/or silencer(s) for tight control of the production of the modified RELA protein encoded by the heterologous polynucleotide present within the gene transfer vector. Indeed, it may be useful to control the production of the modified RELA protein to ensure sufficient production. The term “genetic construct” or “gene transfer vector” encompasses nucleic acid particles, like mRNA, mRNa-nanoparticles, that encode the RELA protein of the invention.

The gene transfer vector may be introduced into the immune cells, in particular T cell(s), by methods known in the art. As examples, the gene transfer vector may be introduced by transduction (in particular by viral transduction when the gene transfer vector is issued or derived from a virus), transfection, in particular with a liposome or a cationic liposome, injection, microinjection or biolistic, in particular with gene transfer vector-coated particles or naked-DNA injection or injection of DNA-polymer conjugates, electroporation, and the like. When the gene transfer vector is a viral particle produced by helper or a production cell line, the introduction of the gene transfer vector into T cell(s) occurs by transduction of T cell(s) with the previously rescued (or harvested) viral particles.

Once the gene transfer vector has been inserted into the immune cells, in particular T cells, the cells are considered “modified”, “engineered”, “transformed”, “genetically modified”, “genetically engineered” or “genetically transformed”. By modified, engineered, transformed, genetically modified, genetically engineered and genetically transformed, it should be understood that the cells comprise the vector or the vector genome containing the polynucleotide encoding. The expression “modified cells” used therein may be replaced by the expression “engineered cells” or with the expression “transformed cells”; these three expressions may be used interchangeably. The cells may be expanded in vitro and/or ex vivo to increase the overall number of cells and/or to select a sub-population thereof. In a particular embodiment of the invention, the cells are T cells and are first expanded in vitro and/or ex vivo after T cells were obtained from an individual, and T cells are modified after. In another embodiment, the T cells are first modified after their recovery from an individual, and then expanded in vitro or ex vivo. In a particular embodiment, T cells are first expanded in vitro and/or ex vivo, then modified, and again expanded in vitro and/or ex vivo. By expansion, it should be understood that the cells are cultivated in vitro or ex vivo, in particular to increase the overall number of T cells. Other step(s) of modifying T cells may also occur in vitro and/or ex vivo, either before and/or after the insertion of the heterologous polynucleotide, for example to initiate differentiation of the cells towards a particular differentiation pathway, or to further modify T cells (for example with CAR or TCR technologies).

The invention also concerns a method for providing engineered immune cells, in particular a lymphocyte, more particularly a T cell by:
Incorporating into immune cells a genetic construct according to any embodiment disclosed herein, in conditions allowing the transcription and/or the production of the RELA protein encoded by the genetic construct. Such incorporation may be performed by transfection of the genetic construct, or by injection of the genetic construct into the immune cells.

A genetic construct according to the invention may further comprise a sequence or a plurality of sequences encoding a Chimeric Antigen Receptor (CAR) for expression into the cells, in particular into T cells, of a CAR along with the modified RELA protein.

A genetic construct according to the invention may be used to engineer immune cells in vivo, ex vivo, or in situ.

Thus, the invention also concerns a genetic construct encoding a modified RELA protein according to the invention, and a Chimeric Antigen Receptor, for modifying immune cells, in particular t cells, to provide CAR-T cells comprising and/or expressing a modified RELA protein.

The invention also concerns the use of a genetic construct encoding a modified RELA protein according to the invention, and a Chimeric Antigen Receptor, for modifying immune cells, in particular t cells, to provide CAR-T cells comprising and/or expressing a modified RELA protein.

The invention also concerns a genetic construct encoding a modified RELA protein according to the invention, and a second, different, genetic construct encoding a Chimeric Antigen Receptor, for modifying immune cells, in particular t cells, to provide CAR-T cells comprising and/or expressing a modified RELA protein.

The invention also concerns the use of a genetic construct encoding a modified RELA protein according to the invention, and a second, different, genetic construct encoding a Chimeric Antigen Receptor, for modifying immune cells, in particular t cells, to provide CAR-T cells comprising and/or expressing a modified RELA protein.

The genetic constructs may be nucleic acid-nanoparticles (mRNA-nanoparticles), encoding the modified RELA protein and/or the chimeric antigen receptor.

The invention also concerns a method for providing engineered immune cells, in particular a lymphocyte, more particularly a T cell, with an enhanced interferon (IFN) metabolism, in particular with an enhanced production of IFN, like IFN-I and/or IFN-III, the method comprising the following step:
Expressing within immune cells a modified RELA protein according to any embodiment disclosed herein to provide engineered immune cells,
Optionally contacting the (engineered) immune cells with a DNA methylation inhibitor, and/or overexpressing IRF3 in the (engineered) immune cells
Selecting immune cells that produces more interferon, in particular IFN-I and/or IFN-III, as compared non-engineered immune cells.

### Pharmaceutical Composition

In another aspect of the invention, it is provided a pharmaceutical composition comprising a modified RELA protein and/or cell comprising and/or expressing a modified RELA protein or comprising a genetic construct encoding such a modified RELA protein, and/or a genetic construct encoding such a modified RELA protein, and a pharmaceutical vehicle. Said pharmaceutical composition can optionally further comprise a different active ingredient. Said composition is provided in particular in a formulation suitable for systemic administration, or for local administration. The pharmaceutical composition is provided in any suitable form for administration, including as a solution, in particular a sterile aqueous solution, as a suspension, as a solid, in particular a lyophilized solid, in particular for adsorption on a patch and/or for resuspension and administration as a solution, as a pill, tablet or other solid form suitable for oral administration. [A composition provided herein may further comprise an additional compound having a therapeutic immunomodulator effect, in particular on immune cells, in particular in T cells.

Additional components may be added within the formulation, the composition or the medicament, for example for enhancing survival of the T cells within their packaging until the formulation, the composition or the medicament is administered to a patient in need thereof. The composition or the formulation may be in particular a pharmaceutical composition or a pharmaceutical formulation. Such a composition may comprise pharmaceutical acceptable components, like but not limited to pharmaceutically suitable excipient or carrier or vehicle, when used for systemic or local administration. A pharmaceutically suitable carrier or vehicle refers to a non-toxic solid, semi-solid or liquid filler, diluent, encapsulating material and formulation like phosphate buffered saline solutions, distilled water, emulsions such as oil/water emulsions, wetting agents and the like, dextrose, saline, ethanol and combinations thereof. The pharmaceutical composition, the pharmaceutical formulation and the medicament may further comprise pharmaceutically acceptable or compatible ingredient. The term “pharmaceutically acceptable or compatible ingredient” refers to a pharmaceutically acceptable diluent, adjuvant, excipient, or vehicle with which modified T cells may be administered. The composition or the formulation or the medicament may be administered by local administration, in particular subcutaneous administration, intro-tumoral administration. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol, and the like. The composition, if desired, can also contain minor amounts of wetting or emulsifying agents, or pH buffering agents. These compositions can take the form of solutions, suspensions, emulsion, capsules, sustained-release formulations and the like. Examples of suitable pharmaceutical carriers are described in “Remington's Pharmaceutical Sciences” by E. W. Martin.

Such compositions will contain a therapeutically effective amount of the compound, typically in purified form, together with a suitable amount of carrier so as to provide the form for proper administration to the patient. The product of the invention, in any form (i.e. cells, modified proteins composition, formulation, medicament), may be administered by injection, by means of a catheter, or by means of an implant, the implant being of a porous, non-porous, or gelatinous material, including a membrane, in particular for controlled-term delivery.

In some embodiments, the cell, the modified RELA protein and/or the pharmaceutical composition according to the invention is administered in combination with additional cancer therapies. In particular, compound and/or pharmaceutical composition of the invention may be administered in combination with targeted therapy, immunotherapy such as immune checkpoint therapy and immune checkpoint inhibitor, co-stimulatory antibodies, chemotherapy and/or radiotherapy.

As used herein, the term “immunotherapy” refers to a cancer therapeutic treatment using the immune system to reject cancer. The therapeutic treatment stimulates the patient's immune system to attack the malignant tumor cells.

Immune checkpoint therapy such as checkpoint inhibitors include, but are not limited to programmed death-1 (PD-1) inhibitors, programmed death ligand-1 (PD-L1) inhibitors, programmed death ligand-2 (PD-L2) inhibitors, lymphocyte-activation gene 3 (LAG3) inhibitors, T-cell immunoglobulin and mucin-domain containing protein 3 (TIM-3) inhibitors, T cell immunoreceptor with Ig and ITIM domains (TIGIT) inhibitors, B- and T-lymphocyte attenuator (BTLA) inhibitors, V-domain Ig suppressor of T-cell activation (VISTA) inhibitors, cytotoxic T-lymphocyte-associated protein 4 (CTLA4) inhibitors, Indoleamine 2,3-dioxygenase (IDO) inhibitors, killer immunoglobulin-like receptors (KIR) inhibitors, KIR2L3 inhibitors, KIR3DL2 inhibitors and carcinoembryonic antigen-related cell adhesion molecule 1 (CEACAM-1) inhibitors. In particular, checkpoint inhibitors include antibodies anti-PD1, anti-PD-L1, anti-CTLA-4, anti-TIM-3, anti-LAG3. Immune checkpoint therapy also include co-stimulatory antibodies delivering positive signals through immune-regulatory receptors including but not limited to ICOS, CD137, CD27, OX-40 and GITR.

Example of anti-PD1 antibodies include, but are not limited to, nivolumab, cemiplimab (REGN2810 or REGN-2810), tislelizumab (BGB-A317), tislelizumab, spartalizumab (PDR001 or PDR-001), ABBV-181, JNJ-63723283, BI 754091, MAG012, TSR-042, AGEN2034, pidilizumab, nivolumab (ONO-4538, BMS-936558, MDX1106, GTPL7335 or Opdivo), pembrolizumab (MK-3475, MK03475, lambrolizumab, SCH-900475 or Keytruda) and antibodies described in International patent applications WO2004004771, WO2004056875, WO2006121168, WO2008156712, WO2009014708, WO2009114335, WO2013043569 and WO2014047350. Example of anti-PD-L1 antibodies include, but are not limited to, LY3300054, atezolizumab, durvalumab and avelumab. Example of anti-CTLA-4 antibodies include, but are not limited to, ipilimumab (see, e.g., US patents U.S. Pat. Nos. 6,984,720 and 8,017,114), tremelimumab (see, e.g., US patents U.S. Pat. Nos. 7,109,003 and 8,143,379), single chain anti-CTLA4 antibodies (see, e.g., International patent applications WO1997020574 and WO2007123737) and antibodies described in US patent U.S. Pat. No. 8,491,895. Example of anti-VISTA antibodies are described in US patent application US20130177557. Example of inhibitors of the LAG3 receptor are described in US patent U.S. Pat. No. 5,773,578. Example of KIR inhibitor is IPH4102 targeting KIR3DL2.

In some embodiments, the compound and/or pharmaceutical composition of the invention may be used in combination with targeted therapy. As used herein, the term “targeted therapy” refers to targeted therapy agents, drugs designed to interfere with specific molecules necessary for tumor growth and progression. For example, targeted therapy agents such as therapeutic monoclonal antibodies target specific antigens found on the cell surface, such as transmembrane receptors or extracellular growth factors. Small molecules can penetrate the cell membrane to interact with targets inside a cell. Small molecules are usually designed to interfere with the enzymatic activity of the target protein such as for example proteasome inhibitor, tyrosine kinase or cyclin-dependent kinase inhibitor, histone deacetylase inhibitor. Targeted therapy may also use cytokines.

Examples of such targeted therapy include with no limitations: Ado-trastuzumab emtansine (HER2), Afatinib (EGFR (HER1/ERBB1), HER2), Aldesleukin (Proleukin), alectinib (ALK), Alemtuzumab (CD52), axitinib (kit, PDGFRbeta, VEGFR1/2/3), Belimumab (BAFF), Belinostat (HDAC), Bevacizumab (VEGF ligand), Blinatumomab (CD19/CD3), bortezomib (proteasome), Brentuximab vedotin (CD30), bosutinib (ABL), brigatinib (ALK), cabozantinib (FLT3, KIT, MET, RET, VEGFR2), Canakinumab (IL-1 beta), carfilzomib (proteasome), ceritinib (ALK), Cetuximab (EGFR), cofimetinib (MEK), Crizotinib (ALK, MET, ROS1), Dabrafenib (BRAF), Daratumumab (CD38), Dasatinib (ABL), Denosumab (RANKL), Dinutuximab (B4GALNT1 (GD2)), Elotuzumab (SLAMF7), Enasidenib (IDH2), Erlotinib (EGFR), Everolimus (mTOR), Gefitinib (EGFR), Ibritumomab tiuxetan (CD20), Sonidegib (Smoothened), Sipuleucel-T, Siltuximab (IL-6), Sorafenib (VEGFR, PDGFR, KIT, RAF), (Tocilizumab (IL-6R), Temsirolimus (mTOR), Tofacitinib (JAK3), Trametinib (MEK), Tositumomab (CD20), Trastuzumab (HER2), Vandetanib (EGFR), Vemurafenib (BRAF), Venetoclax (BCL2), Vismodegib (PTCH, Smoothened), Vorinostat (HDAC), Ziv-aflibercept (PIGF, VEGFA/B), Olaparib (PARP inhibitor).

In some embodiments, the compound and/or pharmaceutical composition of the invention may be used in combination with chemotherapy. As used herein, the term “antitumor chemotherapy” or “chemotherapy” has its general meaning in the art and refers to a cancer therapeutic treatment using chemical or biochemical substances, in particular using one or several antineoplastic agents or chemotherapeutic agents. Chemotherapeutic agents include, but are not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; methylhydrazine derivatives including N-methylhydrazine (MIH) and procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-1 1); topoisomerase inhibitor RFS 2000; difluoromethylomithine (DMFO); retinoids such as retinoic acid; capecitabine; anthracyclines, nitrosoureas, antimetabolites, epipodophylotoxins, enzymes such as L-asparaginase; anthracenediones; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide; and pharmaceutically acceptable salts, acids or derivatives of any of the above.

In some embodiments, the compound and/or pharmaceutical composition of the invention is administered to the patient in combination with radiotherapy. Suitable examples of radiation therapies include, but are not limited to external beam radiotherapy (such as superficial X-rays therapy, orthovoltage X-rays therapy, megavoltage X-rays therapy, radiosurgery, stereotactic radiation therapy, Fractionated stereotactic radiation therapy, cobalt therapy, electron therapy, fast neutron therapy, neutron-capture therapy, proton therapy, intensity modulated radiation therapy (IMRT), 3-dimensional conformal radiation therapy (3D-CRT) and the like); brachytherapy; unsealed source radiotherapy; tomotherapy; and the like. Gamma rays are another form of photons used in radiotherapy. Gamma rays are produced spontaneously as certain elements (such as radium, uranium, and cobalt 60) release radiation as they decompose, or decay. In some embodiments, radiotherapy may be proton radiotherapy or proton minibeam radiation therapy. Proton radiotherapy is an ultra-precise form of radiotherapy that uses proton beams (Prezado Y, Jouvion G, Guardiola C, Gonzalez W, Juchaux M, Bergs J, Nauraye C, Labiod D, De Marzi L, Pouzoulet F, Patriarca A, Dendale R. Tumor Control in RG2 Glioma-Bearing Rats: A Comparison Between Proton Minibeam Therapy and Standard Proton Therapy. Int J Radiat Oncol Biol Phys. 2019 Jun. 1; 104(2):266-271. doi: 10.1016/j.ijrobp.2019.01.080; Prezado Y, Jouvion G, Patriarca A, Nauraye C, Guardiola C, Juchaux M, Lamirault C, Labiod D, Jourdain L, Sebrie C, Dendale R, Gonzalez W, Pouzoulet F. Proton minibeam radiation therapy widens the therapeutic index for high-grade gliomas. Sci Rep. 2018 Nov. 7; 8(1):16479. doi: 10.1038/s41598-018-34796-8). Radiotherapy may also be FLASH radiotherapy (FLASH-RT) or FLASH proton irradiation. FLASH radiotherapy involves the ultra-fast delivery of radiation treatment at dose rates several orders of magnitude greater than those currently in routine clinical practice (ultra-high dose rate) (Favaudon V, Fouillade C, Vozenin MC. The radiotherapy FLASH to save healthy tissues. Med Sci (Paris) 2015; 31: 121-123. DOI: 10.1051/medsci/20153102002); Patriarca A., Fouillade C. M., Martin F., Pouzoulet F., Nauraye C., et al. Experimental set-up for FLASH proton irradiation of small animals using a clinical system. Int J Radiat Oncol Biol Phys, 102 (2018), pp. 619-626. doi: 10.1016/j.ijrobp.2018.06.403. Epub 2018 Jul. 11).

Combination of a Modified RELA Protein with an Inhibitor of the DNA Methylation and/or with IRF3 Overexpression

According to an embodiment of the invention, it is provided a combination of product comprising:
A modified RELA protein according to at least one embodiment disclosed herein; the protein may be present within an immune cell; and
An inhibitor of the DNA methylation, in particular a DNA methyltransferase inhibitor (DNMTi), more particularly decitabine or azacytidine, most particularly azacytidine (5AZA).

Such a combination is particularly useful for enhancing the interferon production in immune cells, in particular in T cells.

According to an embodiment of the invention, it is provided a combination of product comprising:
An immune cell according to the invention (i.e. an immune cell that comprises and/or expresses a modified RELA protein derived or issued from a wild-type RELA protein, in particular a wild-type human RELA protein, more particularly a wild-type human RELA protein of SEQ ID No. 1, and/or that comprises a genetic construct encoding such a modified RELA protein), and
An inhibitor of the DNA methylation, in particular a DNA methyltransferase inhibitor (DNMTi), more particularly decitabine or azacytidine, most particularly azacytidine (5AZA).

According to an embodiment of the invention, it is provided a combination of product comprising:
a genetic construct encoding a modified RELA protein according to the invention, and
An inhibitor of the DNA methylation, in particular a DNA methyltransferase inhibitor (DNMTi), more particularly decitabine or azacytidine, most particularly azacytidine (5AZA).

According to an embodiment of the invention, it is provided a combination of product comprising:
A modified RELA protein according to at least one embodiment disclosed herein; the protein may be present within an immune cell or inserted within an immune cell; and
A genetic construct encoding IRF3 (Interferon Regulatory Factor 3); the genetic construct allowing an immune cell according to the invention to express IRF3. The genetic construct encoding IRF3 may correspond to the genetic construct already disclosed herein, provided that it comprises a polynucleotide sequence that encodes IRF3.

Such a combination is particularly useful for enhancing the interferon production in immune cells, in particular in T cells.

According to an embodiment of the invention, it is provided a combination of product comprising:
An immune cell according to the invention (i.e. an immune cell that comprises and/or expresses a modified RELA protein derived or issued from a wild-type RELA protein, in particular a wild-type human RELA protein, more particularly a wild-type human RELA protein of SEQ ID No. 1, and/or that comprises a genetic construct encoding such a modified RELA protein), and
a genetic construct encoding IRF3.

According to an embodiment of the invention, it is provided a combination of product comprising:
a genetic construct encoding a modified RELA protein according to the invention, and
a genetic construct encoding IRF3.

According to an embodiment of the invention, it is provided a combination of product comprising:
An immune cell according to the invention (i.e. an immune cell that comprises and/or expresses a modified RELA protein derived or issued from a wild-type RELA protein, in particular a wild-type human RELA protein, more particularly a wild-type human RELA protein of SEQ ID No. 1, and/or that comprises a genetic construct encoding such a modified RELA protein),
An inhibitor of the DNA methylation, in particular a DNA methyltransferase inhibitor (DNMTi), more particularly decitabine or azacytidine, most particularly azacytidine (5AZA); and
a genetic construct encoding IRF3.

According to an embodiment of the invention, it is provided a combination of product comprising:
a genetic construct encoding a modified RELA protein according to the invention,
An inhibitor of the DNA methylation, in particular a DNA methyltransferase inhibitor (DNMTi), more particularly decitabine or azacytidine, most particularly azacytidine (5AZA), and
a genetic construct encoding IRF3.

According to an embodiment of the invention, it is provided a combination of product comprising:
A modified RELA protein according to at least one embodiment disclosed herein; the protein may be present within an immune cell or inserted within an immune cell,
A genetic construct encoding IRF3 (Interferon Regulatory Factor 3); the genetic construct allowing an immune cell according to the invention to express IRF3. The genetic construct encoding IRF3 may correspond to the genetic construct already disclosed herein, provided that it comprises a polynucleotide sequence that encodes IRF3,
a genetic construct encoding IRF3.

### Use of the Product of the Invention in Therapies

In an aspect of the invention, it is provided a method of treating a patient having a cancer, the method comprising administering to the individual a therapeutically effective amount of:
A modified RELA protein according to any embodiment disclosed herein; and/or
An immune cell comprising or expressing a modified RELA protein according to any embodiment disclosed herein and/or comprising a genetic construct encoding a modified RELA protein according to any embodiment disclosed herein; and/or
a genetic construct encoding a modified RELA protein according to any embodiment disclosed herein; and/or
a pharmaceutical composition comprising a modified RELA protein according to the invention, either within a cell or not, and/or an immune cell comprising a modified RELA protein according to any embodiment disclosed herein and/or comprising a genetic construct encoding a modified RELA protein according to any embodiment disclosed herein, and/or a genetic construct encoding a modified RELA protein according to any embodiment disclosed herein.

As used herein, the term “subject” or “patient” denotes a mammal, such as a rodent, a feline, a canine, and a primate. Preferably, a subject according to the invention is a human being.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. The term “treatment” encompasses the prophylactic treatment. As used herein, the term “prevent” refers to the reduction in the risk of acquiring or developing a given condition.

The product according to any embodiment disclosed herein may be used in therapy, in particular immunotherapy, to treat or prevent various types of diseases including, but not limited to, cancers or infectious diseases. Cancers that may be targeted accordingly include cancers with a solid tumor, cancers with a liquid tumor, melanoma, ovarian cancers, breast cancers, colorectal cancers, recurrent cancers. In some aspects, the disease or condition to be treated or prevented is a cancer or associated symptom. In an aspect of the invention, a product according to any embodiment disclosed herein is provided for use in the treatment of a cancer. A cancer is a disease involving abnormal cell growth with the potential to invade or spread to other parts of the body. According to the invention, the cancer which affects or affected a patient may be selected from the list consisting of bladder cancer, bone cancer, brain cancer, breast cancer, cervical cancer, colon cancer, esophageal cancer, gastric cancer, head & neck cancers, hodgkin's lymphoma, leukemia, liver cancer, lung cancer, melanoma, mesothelioma, multiple myeloma, myelodysplastic syndrome, non-hodgkin's lymphoma, ovarian cancer, pancreatic cancer, prostate cancer, rectal cancer, renal cancer, sarcoma, skin cancer, testicular cancer, thyroid cancer or uterine cancer. In a particular embodiment, the cancer which affect or affected a patient is a lung cancer, more particularly a lung carcinoma.

The cells modified and expanded according to the invention may thereafter be used in immunotherapy, such as adoptive cell therapy. Adoptive cell therapy involves the transfer of autologous or allogenic immune cells, in particular antigen-specific or pathogen-specific immune cells, the properties of which are changed ex vivo, to a patient in need thereof. As used herein, a patient is in particular a human patient, and a disease is in particular a human disease. In autologous therapy, immune cells are recovered from a patient, genetically modified in vitro or ex vivo according to the invention, possibly selected in vitro or ex vivo, and/or further modified to change their properties and/or functions to target diseased cells when administered in vivo and are expanded (or cultured) in vitro or ex vivo for amplifying the number of immune cells when necessary, and finally infused into the patient. Alternatively, the immune cells may be used in an allogenic immunotherapy. Allogenic therapy encompasses obtaining immune cells from individual(s) belonging to the same species of the patient, but genetically dissimilar, and administering these cells after modifying them according to the invention to a patient.

In an aspect, it is provided a method of increasing the killing of tumor cells, the method comprising administering to the individual a therapeutically effective amount of:
a modified RELA protein according to any embodiment disclosed herein; and/or
an immune cell comprising and/or expressing a modified RELA protein according to any embodiment disclosed herein and/or comprising a genetic construct encoding a modified RELA protein according to any embodiment disclosed herein; and/or
a genetic construct encoding a modified RELA protein according to any embodiment disclosed herein; and/or
a pharmaceutical composition comprising a modified RELA protein according to the invention, either within a cell or not, and/or an immune cell comprising and/or expressing a modified RELA protein according to any embodiment disclosed herein and/or comprising a genetic construct encoding a modified RELA protein according to any embodiment disclosed herein, and/or a genetic construct encoding a modified RELA protein according to any embodiment disclosed herein.

In an aspect of the invention, it is provided a method of treating an infectious disease in an individual in need thereof, the method comprising administering to the individual a therapeutically effective amount of:
a modified RELA protein according to any embodiment disclosed herein; and/or
an immune cell comprising and/or expressing a modified RELA protein according to any embodiment disclosed herein and/or comprising a genetic construct encoding a modified RELA protein according to any embodiment disclosed herein; and/or
a genetic construct encoding a modified RELA protein according to any embodiment disclosed herein; and/or
a pharmaceutical composition comprising a modified RELA protein according to the invention, either within a cell or not, and/or an immune cell comprising and/or expressing a modified RELA protein according to any embodiment disclosed herein and/or comprising a genetic construct encoding a modified RELA protein according to any embodiment disclosed herein, and/or a genetic construct encoding a modified RELA protein according to any embodiment disclosed herein.

An infectious disease may be a chronic infection like a chronic viral infection. The infectious disease may be caused by a virus, in particular infected by a retrovirus or a lentivirus, more particularly infected by a HIV, like HIV-I or HIV-II.

In an aspect, it is provided a method of enhancing a T cell response in an individual in need thereof, comprising administering to the individual a therapeutically effective amount of:
a modified RELA protein according to any embodiment disclosed herein; and/or
an immune cell comprising and/or expressing a modified RELA protein according to any embodiment disclosed herein and/or comprising a genetic construct encoding a modified RELA protein according to any embodiment disclosed herein; and/or
a genetic construct encoding a modified RELA protein according to any embodiment disclosed herein; and/or
a pharmaceutical composition comprising a modified RELA protein according to the invention, either within a cell or not, and/or an immune cell comprising and/or expressing a modified RELA protein according to any embodiment disclosed herein and/or comprising a genetic construct encoding a modified RELA protein according to any embodiment disclosed herein, and/or a genetic construct encoding a modified RELA protein according to any embodiment disclosed herein.

In an aspect, it is provided a method of increasing the resistance of immune cells to an infectious disease, in particular an infectious disease caused by a virus, in particular infected by a retrovirus or a lentivirus, more particularly infected by a HIV, like HIV-I or HIV-II, the method comprising administering to the individual a therapeutically effective amount of:
a modified RELA protein according to any embodiment disclosed herein; and/or
an immune cell comprising and/or expressing a modified RELA protein according to any embodiment disclosed herein and/or comprising a genetic construct encoding a modified RELA protein according to any embodiment disclosed herein; and/or
a genetic construct encoding a modified RELA protein according to any embodiment disclosed herein; and/or
a pharmaceutical composition comprising a modified RELA protein according to the invention, either within a cell or not, and/or an immune cell comprising and/or expressing a modified RELA protein according to any embodiment disclosed herein and/or comprising a genetic construct encoding a modified RELA protein according to any embodiment disclosed herein, and/or a genetic construct encoding a modified RELA protein according to any embodiment disclosed herein.

In an aspect, it is provided a method of increasing the cytokine production in immune cells, in particular in immune cells of a patient having a cancer or an infectious disease, the method comprising administering to the individual a therapeutically effective amount of:
a modified RELA protein according to any embodiment disclosed herein; and/or
an immune cell comprising and/or expressing a modified RELA protein according to any embodiment disclosed herein and/or comprising a genetic construct encoding a modified RELA protein according to any embodiment disclosed herein; and/or
a genetic construct encoding a modified RELA protein according to any embodiment disclosed herein; and/or
a pharmaceutical composition comprising a modified RELA protein according to the invention, either within a cell or not, and/or an immune cell comprising and/or expressing a modified RELA protein according to any embodiment disclosed herein and/or comprising a genetic construct encoding a modified RELA protein according to any embodiment disclosed herein, and/or a genetic construct encoding a modified RELA protein according to any embodiment disclosed herein.

### Examples Illustrating the Invention

### Material and Method

### Primary Cells Isolation and Culture:

Plasmapheresis blood pockets were obtained from healthy adult volunteers after informed consent from the EFS (Establishment of French blood collection). Peripheral blood mononuclear cells (PBMCs) were isolated using Ficoll-Paque Plus (GE-17144003) and left overnight at 4° C. in RPMI (Thermo fischer #61870-010) with 10% heat-decomplemented fetal bovine serum (FBS, Eurobio #CVFSVFO0-01). CD14+ cells were enriched by positive selection (Miltenyi #130-050-201), the CD14 negative fraction was subsequently used to isolate total CD4+ T cells by negative selection (Stem Cell #17952). CD14+ cells were cultured in RPMI containing 10% FBS, 5% Penicillin-Streptomycin (PS, Thermo Fisher #10378-016), 50 μg/ml Gentamicin (Thermo Fisher #15750-045) and 10 mM HEPES (Thermo Fisher #15630-056). CD14+ cells were differentiated ex vivo into MDDC by supplementing the culture media with 50 ng/ml IL-4 (Miltenyi #130-093-922) and 10 ng/ml GM-CSF (Miltenyi #130-093-867). MDDC were used for experiments 3 or 4 days following the start of the culture. CD14+ cells were differentiated ex vivo into macrophages by adding 50 ng/ml MCSF (Miltenyi #130-096-492) to the culture media containing RPMI with 1% PS, 5% FBS and 5% human serum (Sigma #H4522). MDM were used 7 days following culture. Freshly isolated CD4+ T cells were cultured in X-VIVO 15 (Lonza #BE02-060F) and activated using anti-CD3 and anti-CD28 Dynabeads (fisher scientific #10548353) at the ratio of 1:5 (bead:cell). 100 U/ml human IL-2 (Immunotools #11340027) was added to cultures 2 days following TCR stimulation and media with IL-2 was replenished every 48 hours. To assess proliferation, freshly isolated CD4+ T cells were stained with cell proliferation dye (thermo fischer #65-0840-85) prior to TCR stimulation.

Peripheral pDC, cDC1 and cDC2 were enriched using pan-DC enrichment kit (Stem cell #19251), and subsequently stained with anti-HLA-DR APCeFluor780, anti-CD1c PerCPeFluor710 (eBioscience), anti-CD123 Viogreen, anti-CD45RA Vioblue (Miltenyi), anti-AXL PE (Clone #108724, R&D Systems) anti-CD33 PE-CF594, anti-CLEC9A PE (BD) and with a cocktail of FITC conjugated antibodies against lineage markers CD19 (Miltenyi), CD3, CD14, CD16 and CD34 (BD) and sorted on a FACSAria as previously described⁶⁶. pDC were defined as: Lin-HLA-DR+CD33− CD45RA+CD123+ AXL−, cDC1 as Lin−HLA−DR+CD33+CD45RA−CD1c− CLECL9A+) as cDC2 as Lin− HLA−DR+CD33+CD45RA−CD1c+ Lin− corresponds to HLA− DR+CD33+CD45RA− CD1c+. Sorted cells were cultured in X-VIVO 15 with 5% PS and 3 μg/ml GM-CSF.

### Plasmids:

pSIV3+, psPAX2, HXB2 env, CMV-VSVG, pTRIP-SFFV-GFP and pTRIP-SFFV-GFPIRF3 were previously described⁶⁷. pTRIP-SFFV-GFP-RELA was obtained by PCR cloning of RELA (addgene #23255) onto the pTRIP-SFFV-GFP background. RELA K5R and RELA K5Q mutants were generated by subcloning DNA fragments (Twist biosciences) into pTRIP-SFFV-GFP-RELA plasmid, resulting in pTRIP-SFFV-GFP-RELA K5R and pTRIP-SFFV-GFP-RELA K5Q. RELA K310R mutant was generated by overlapping PCR mutagenesis in pTRIP-SFFV-GFPRELA plasmid resulting in pTRIP-SFFV-GFP-RELA K310R. pLKO.1-puro-IRF7sh1 (IRF7sh1, CCCGAGCTGCACGTTCCTATA, SEQ ID No. 18), pLKO.1-purolRF7sh5 (IRF7sh5, CGCAGCGTGAGGGTGTGTCTT, SEQ ID No. 19) and pLKO.1puro-shLacZ (LacZsh) were previously described⁶⁸. HIV-mTagBFP2 and HIV-2 ROD9 AenvAnef mTagBFP2+ were previously⁶⁹. pTRIP-SFFV-GFP (Ctrl), pTRIP-SFFV-EGFP-FLAG-cGAS (cGAS), pTRIP-SFFV-EGFP-NLS (NLS-Ctrl) pTRIP-SFFV-EGFP-NLS-FLAG-cGAS (NLS-cGAS) were previously described⁶³. Human CD19 cDNA (Genescript, NM_001178098) was cloned into the pCDH-CMV-MCS-EF1-Puro plasmid (System Biosciences) to create pCDH-CMV-CD19 puro.

### Lentivirus Production:

293FT cells were cultured in DMEM (Thermo Fisher #61965026) with 5% PS and 10% FBS. 293FT cells were plated at 0.8 million cells in 6-well plate and transfected with 3 μg of DNA complexed in 8 μl of TransIT-293 (Mirus Bio #MIR2706) per well. The ratio of DNA used for transfections was as follows 0.2 μg HXB2 env, 0.2 μg CMV-VSVG, 1 μg psPAX2 and 1.6 μg of pTRIP-SFFV or pLKO.1 lentivector. The ratio of plasmids for the production of HIV-1 and HIV-2 BFP single-round reporter viruses were 0.2 μg HXB2 env, 0.2 μg CMV-VSVG and 2.6 μg HIV plasmid. SIV-VLPs were produced using 0.4 μg CMV-VSVG with 2.6 μg pSIV3+. Lentiviruses for MDM were prepared by plating 7 millions 293FT in T75 flasks and transfected with 8.35 μg DNA complexed in 116 μl PEImax (0.75 mM) (Polyscience #24765) per flask. The ratio of DNA used for transfection includes 3 μg psPAX2, 1.25 μg CMV-VSVG and 4.10 μg pTRIP-SFFV-GFP. For SIV-VLP production for MDM, 2.5 μg CMV-VSVG with 8.2 μg pSIV3+ were used. 18 hours following transfection media was removed and replenished with fresh media (3 ml for T cells and MDDC or 8.5 ml for MDM). 24 to 26 hours later, viral supernatants were harvested, filtered using 0.45 μM filters and used fresh or stored at −80° C.

For CAR expression, rLV.EFA.19BBz CAR lentivirus was produced using pLV plasmid, pHIV-GagPol and pEnv and concentrated by ultracentrifugation (Flash therapeutics). Titer was determined by serial dilution on activated human T cells.

### Crispr-Cas9 Nucleofections:

24 hours post TCR stimulation, 2 million cells washed with PBS and nucleofected using a Lonza kit (#V4XP-3032). Cr-RNA and TracrRNA (IDT #1072534) were annealed by heating at 95° C. for 5 minutes. Two gRNAs targeting cGAS were used, 100 pmol of each gRNA was incubated separately with 66 pmol of Cas9 protein (IDT #1081059) for 10 mins at RT to form complexes. The two complexes were combined, and a final volume of 5 ul containing 200 pmol of guides and 135 pmol of Cas9 was used per reaction. Nucleofection was performed using EH-100 program using the 4D-Nucelofector (Lonza). Following nucleofection cells were cultured for 3 days in 300 U/ml IL-2. 72 hours following nucleofection, cells were harvested to assess efficiency of gene knockout. cGAS gRNA targeted the following genomic sequences AGACTCGGTGGGATCCATCG (SEQ ID No. 20, IDT #Hs.Cas9.MB21 D1.1.AA) and CGAAGCCAAGACCTCCGCCC (SEQ ID No. 21, IDT #Hs.Cas9.MB21D1.1.AL). Nucleofections were performed 5 to 6 hours prior to lentivirus transductions.

### Lentivirus Transductions:

CD14+ cells were plated at 1 million cells/ml and transduced with equal volumes of freshly harvested SIV-VLPs and pTRIP-SFFV vectors in the presence of 8 μg/ml protamine (Sigma #P4020). CD4+ T cells were transduced with lentivectors 24 hours post-TCR stimulation in 100 μl of cells (0.2 million cells) and 100 μl of freshly harvested lentivirus in the presence of 8 μg/ml protamine. T cells were spinoculated at 1200 g for 2 hours at 25° C. 0.5 million THP-1 cells in 500 μl media (RPMI 10% FBS 1% PS) were transduced with 500 μl freshly harvested lentivirus in the presence of 8 μg/ml protamine. 1 μg/ml Puromycin (Invivogen #ant-pr-1) was added 2 days post-transduction when cells were transduced with pLKO.1-puro plasmid.

### Primary Cell Stimulations and Treatments:

For stimulation of STING by extracellular ligands, 2.5 μg of ADUS100 (invivogen #tlrl-nacda2r-01) or 2′3′-cGAMP (cGAMP, invivogen #tlrl-nacga23-02) was added to 100,000 cells in 100 μl media either on freshly isolated primary cells or 3-4 days post differentiation for MDDC and 3 days post-TCR stimulation for CD4+ T cells. Cells and supernatant were harvested 18-24 hours post stimulation. For delivery of STING ligands after permeabilization, T cells were used 3 or 4 days post TCR stimulation and MDDC were used 3-4 days following differentiation. 150,000 cells were plated in round bottom 96-well plate. Media was removed by centrifugation and 20 μl 2× permeabilization buffer (100 mM HEPES, 200 mM KCl, 6 mM MgCl2, 0.2 mM DTT, 170 mM Sucrose, 2 mM ATP, 2 mM GTP, 0.4% bovine serum albumin (BSA), 20 μg/ml digitonin) and 20 μl cGAMP (12 μg/ml) was added to cells. 18 to 20 hours following stimulation supernatants and cells were harvested. Cells were stained with a fixable viability dye (Invitrogen #65-0865-14) and acquired on a FACSVerse (BD).

For cGAS stimulation, 2 μg of control GFP plasmid DNA (provided in the kit) was nucelofected in 2 million T cells 72 hours post TCR stimulation (Lonza #VPA-1002) using the programme T020 (Lonza #Nucleofector 2b). Cells were harvested at 4 hours and 24 hours after nucleofection for analysis.

For experiments with conditioned media, CD4+ T cell- and MDDC-conditioned media refer to 0.45 μM filtered conditioned media from cultures of activated CD4+ T cell and MDDC, respectively. CD4+ T cells were pretreated with 100 μl of MDDC-conditioned media or of fresh MDDC culture media, 24 hours prior to cGAMP stimulation. MDDC were pretreated with 100 μl of CD4+ T cell-conditioned media, or of fresh CD4+ T cell culture media, 24 hours prior to cGAMP stimulation. Where indicated, 10 ng/ml recombinant IFN-γ□b (miltenyi #130-096-484) was added.

Where indicated, cells were treated with 5-Azacytidine (5AZA, Sigma #A2385) 48 hours prior to cGAMP stimulation. Fresh 5AZA was added daily to cells, and media was replenished with 5AZA following cGAMP stimulation. 100 nM Trichostatin A (TSA) (sigma #T1952) was added to cells following cGAMP stimulation and left overnight. 25 μM Azidothymidine (AZT, sigma #A2169) and 10 μM nevirapine (NVP, sigma #SML0097) were added prior to lentiviral transduction and replenished when cells were expanded. B118R supernatant and control supernatant were produced from baculovirus-infected cells as described ⁷⁰. 10% of supernatants were added to T cell culture media 2 days after transduction, fresh supernatants were added every day for the next 2 days and maintained overnight after cGAMP stimulation. Where indicated, 1000 U/ml IFNα2a (Immunotools #11343506) was added 20 to 24 hours prior to cGAMP stimulation.

### IFN Quantification:

Culture supernatants were stored at −80° C. and thawed prior to quantification. Concentrations of IFNα2, IFNβ, IFNγ, IFNλ1 and IFNλ2/3 were measured using LEGENDplex Human Type ½/3 Interferon Panel (BioLegend #740396) according to manufacturer's protocol. Data were acquired on a BD FACSVerse (BD) and analyzed with LEGENDplex Software (BioLegend). In figures, dotted lines indicate limit of detection.

### Gene Expression Analysis:

RNA was extracted from 0.4-1 million cells using the Nucleospin RNA II kits (Macherey-Nagel #740955.50). cDNA was synthesized using random primers (invitrogen #48190-011) from 0.1 μg RNA using Superscript III Reverse transcriptase (fisher scientific #18080044). Real-time qPCR (RT-qPCR) was carried out in 20 μl reactions using SYBR Green I Master (Roche #4887352001). Primers used were as follows:

| | IFNL1 |
|---|---|
| | Fwd- |
| (SEQ ID No. 22) | |
| | GGTGACTTTGGTGCTAGGCT, |
| | Rev- |
| (SEQ ID No. 23) | |
| | TGAGTGACTCTTCCAAGGCG), |
| | IFNB |
| | Fwd- |
| (SEQ ID No. 24) | |
| | GTCTCCTCCAAATTGCTCTC, |
| | Rev- |
| (SEQ ID No. 25) | |
| | ACAGGAGCTTCTGACACTGA), |
| | ACTB |
| | Fwd- |
| (SEQ ID No. 26) | |
| | GGACTTCGAGCAAGAGATGG, |
| | Rev- |
| (SEQ ID No. 27) | |
| | AGCACTGTGTTGGCGTACAG), |
| | B2M |
| | Fwd- |
| (SEQ ID No. 28) | |
| | TCTCTGCTGGATGACGTGAG, |
| | Rev- |
| (SEQ ID No. 29) | |
| | TAGCTGTGCTCGCGCTACT). |

### Bulk RNAseq Data Analysis:

Five hours following cGAMP (6 μg/ml) stimulation of activated CD4+ T cells, total RNA was extracted (Macherey-Nagel #740955.50). RNA integrity was verified using Agilent Bioanalyzer (Agilent RNA 6000 Nano kit #5607-1511), all samples had a RIN >9. RNA sequencing libraries were prepared from 500 ng of total RNA using the Illumina TruSeq Stranded mRNA Library preparation kit. A first step of polyA selection using magnetic beads was performed to focus sequencing on polyadenylated transcripts. After fragmentation, cDNA synthesis was performed and resulting fragments were used for dA-tailing and ligated to the TruSeq indexed adapters. PCR amplification was performed to create the final cDNA library (with 13 cycles). After quantification of PCR products, sequencing was carried out using 2*100 cycles (paired-end reads, 100 nucleotides) on a Novaseq6000 instrument, targeting 25M clusters. The data was aligned to the hg19 (ENSEMBL annotation: v.74) genome using the RNA-seq pipeline of the Curie bioinformatics platform, rnaseq v3.1.1. Reads were trimmed with TrimGalore (v.0.6.2) and aligned on the reference genome using STAR (v 2.6.1) ⁷¹. Quality control was done with picard (v.2.18.15), RSeQC (v.2.6.4) and preseq (v.2.0.3)⁷². Read counts were generated with STAR. Quality reports were generated with MultiQC (v.1.7). We filtered the count matrix only keeping genes that have in at least one sample a TPM (transcripts per million) value of 1, this strategy left us with 10671 genes tested of a total of 57820 genes in the count matrix. Differential expression analysis was performed using DESeq2 (1.26.0), complete list of differentially expressed genes is provided in Table S1. ⁷³. A gene was designated as differentially expressed with an adjusted p-value of <0.05 and an absolute log fold change >1. A list of 625 interferon-stimulated gene (ISG) were used for annotation ^{74, 75}. Additionally Bioconductor package clusterProfiler (3.14.3) was used for the pathway over-representation analysis using public databases GO and Kegg⁷⁶. The analysis was run individually on differentially expressed genes (either upregulated or downregulated). Pathways with an adjusted pvalue <0.05 and that contained at least 5 genes from our dataset were considered significant.

### Protein Isolation and Western Blot:

0.5 million cells were lysed for 30 minutes on ice in 50 μl RIPA buffer (50 mM Tris HCl, 150 mM NaCl, 0.1% SDS, 0.5% DOC, 1% NP-40) with Protease inhibitor (Roche #1187358001). To evaluate, phospho-proteins 50 mM NaF and 1 mM sodium orthovanadate were added to the lysis buffer. Protein lysates were cleared by centrifugation at 12,000 g for 10 to 12 minutes at 4° C. 10 μl of 6× Laemmli buffer (fisher scientific #J61337) was added to 50 μl protein lysates and samples were boiled at 95° C. for 15 minutes. Protein samples were resolved on 4%-20% Biorad precast SDS-PAGE gels (#5671125) and transferred onto PVDF membranes (BioRad #1704157). Membranes were blocked in 5% non-fat dry milk in PBS 0.1% tween or in TBS 5% BSA 0.1% Tween to detect phosphorylated proteins. Primary antibodies were used at 1:1000:phospho-STING (clone-D7C3S), STING (clone-D2P2F), serine 172 phospho-TBK1 (clone-D52C2), TBK1 (clone-D11B4), serine 396 phospho IRF3 (clone-D6O1M), IRF3 (clone-D83B9), RELA (clone-C22B4), IRF7 (clone-12G9A36), cGAS (clone-D1 D3G), Actin (clone-C4), Sendai viral proteins (Biozol #MBL-PD029). Secondary anti-rabbit or anti-mouse antibody were used at 1:10,000 (santa cruz #sc-2357, #sc-516102). ECL signal (Biorad #1705061 and #1705062) was recorded on a ChemiDoc Touch Biorad Imager. Data was analyzed and quantified with Image Lab software (Biorad). In figures, dotted lines indicate reordering of lanes from a single original membrane for visualization purpose.

### Confocal Microscopy and Analysis:

4 to 5 days following transduction, 500,000 cells were plated on 0.1% poly-L lysine in water (Sigma #8920) coated coverslips and left for 30 minutes to attach. Cells were fixed in PBS 4% paraformaldehyde for 20 minutes at room temperature (RT). Coverslips were washed twice in PBS and quenched with 500 μl of freshly prepared PBS Glycine (375 mg Glycine in 50 mL of PBS) for 10 minutes at room temperature. Cells were then permeabilized and simultaneously blocked with PBS 0.2% BSA 0.05% saponin 1% goat serum (Sigma #G9023) for 30 minutes at RT. Coverslips were incubated with primary antibody against cGAS (clone D1 D3G) or isotype control (clone DA1E) at a concentration of 0.085 μg/ml in PBS 0.2% BSA 0.05% saponin and left overnight at 4° C. in a moist chamber. Coverslips were washed 5 times with PBS 0.2% BSA 0.05% saponin and incubated in secondary antibody goat anti-Rabbit IgG at 1:400 (Invitrogen #A21246) for 45 minutes at RT. Coverslips were washed 5 times with PBS 0.2% BSA 0.05% saponin, rinsed in water and mounted on a glass slide using 10 μl fluoromount-G with DAPI (Invitrogen #00-4959-520). Glass slides were allowed to dry in a 37° C. chamber for 30 minutes and stored at 4° C. Images were acquired on a Leica Dml8 inverted microscope equipped with an SP8 confocal unit using a 40× (1.3 NA) oil objective. Image analysis was performed using Fiji software⁷⁷. Homemade scripts were used to analyse cGAS localization. All images were smoothened using a filtering of mean radius 1 pixel. Briefly, for each condition a binary mask of the nuclei and a mask of the cell were obtained by applying a threshold respectively on the DAPI signal or the cGAS signal. Thresholding cGAS signal provided more accurate cell contour than thresholding GFP signal due to high variability in the GFP expression. Cells were defined as >20 μm² with a nucleus >16 μm². Average cGAS and GFP intensities were measured in the whole cell, the nuclei and the cytosol (defined by the whole cell excluding the nuclear region).

cGAMP Isolation and Quantification:

Cell pellets were stored at −80° C. until extraction. Frozen pellets were fixed in 80% cold methanol and subjected to 5 freeze-thaw cycles in liquid nitrogen. Samples were then centrifuged at 16,000 g for 20 minutes at 4° C. The supernatants were further subjected to drying in Savant DNA Speed Vac DNA 110 at 65° C. for 2 hours. The pellets were resuspended in 100 μl RNase- and DNase-free water and cGAMP was quantified by ELISA as per manufacturers protocol (Interchim #501700). cGAMP quantity was normalized to cell numbers and represented as pg/million cells.

### Viral Infections:

For HIV infections, 4 days after lentivirus transduction and 2 days following 5AZA treatment, 0.07 million cells in 70 μl media were infected with 70 μl BFP-reporter single-round HIV-1 or HIV-2 with 8 μg/ml protamine and fresh 5′AZA (2 μM). Serial dilutions of viruses at 1/3 were performed. GHOST X4R5 cells were infected in parallel to control the viral titer of the inoculum. Cells and supernatants were harvested 48 hours post-infection.

For Sendai virus infections, 100,000 cells in 100 μl were infected with 100 μl of Sendai virus 200 HA/ml (Charles River, Cantell Strain) with 8 μg/ml protamine. Culture supernatants for IFN quantification were harvested 18-24 hours post-infection. Cells for RNA and protein extraction were harvested 6 hours following infection.

### CAR-T Tumor Spheroid Killing Assay:

CD19+ mKate2+A549 cells were produced by lentiviral transduction using pCDH-CMV-CD19 puro and ILV-EF1a-mKate-9-X01 (Flash therapeutics) lentivectors. 1,000 cells were plated 4 days prior to co-culture in low-cluster 96 well plates (Costar #7007) and allowed to form spheroids in DMEM 10% FBS 5% PS. Total CD3+ T cells were isolated by negative selection from PBMCs (Stemcell #17951) and stimulated with Dynabeads at a ratio of 1:3 (beads:cell). Cells were cultured at 1 million per ml in X-VIVO 15 with 5% PS, 5% decomplemented human serum (Sigma) and 50 μM 2-mercaptoethanol (GIBCO #31350-010). 24 hours following TCR stimulation 100,000 cells in 100 μl were co-transduced with 100 μl of lentivector viral particles (pTRIP-SFFV-GFP or pTRIP-SFFV-GFPRELAK5R) combined with the CAR lentivirus (rLV.EFA.19BBz, Flash therapeutics) at a multiplicity of infection (MOI) of 10 (corresponding usually to less than 1 μl). Spinoculation was carried out at 1200 g for 2 hours at 25° C. Cells were expanded for 48 hours with fresh media and 300 U/ml IL-2. 3 days following transduction T cells were stained with CD19 CAR detection reagent (miltenyi 130-115-965) for 15 minutes at 4° C., washed twice and subsequently with secondary antibody (invitrogen #S21374) for 30 minutes at 4° C. Cells were fixed and acquired on a FACSVerse (BD). The next day, Dynabeads were removed by magnetic separation and 250, 500 or 1,000 CAR+ cells were added per well of A549 spheroids in quadruplicates. Plates were then placed in an IncuCyteS3 and images were acquired using a 10× objective (1.24 μM/pixel) every 3 hours for 5 to 6 days. Images were analysed using the IncuCyte S3 software. At the end of the culture, supernatants were harvested and assayed for IFN concentrations.

### Statistical Analysis:

Data was analysed using PRISM software (version 9, GraphPad Prism). Statistical tests and parameters including statistical significance are reported in Figures Legends. Asterisks denote statistical significance not significant (ns) p>0.05, *p≤0.05, **p≤0.01, ***p≤0.001, ****p≤0.0001.

### Results

### Restricted IFN-I/III Production in Human CD4+ T Cells Benchmarked to Dendritic Cells (FIG. 1)

To assess the IFN response of human blood CD4+ T cells to STING stimulation, we benchmarked their response to donor-matched human blood dendritic cell (DCs) subsets. Stimulation with soluble synthetic STING ligands, namely 2′3′-cGAMP (cGAMP) or the CDN analog and clinical candidate ADU-S100 ²⁶, revealed pDC as the dominant IFN-III and IFN-I producers followed by cDC2 and cDC1 subsets (FIG. 1B). The IFN response to STING ligands in CD4+ T cells was undetectable. Key signaling proteins STING, TBK1, IRF3 and RELA were expressed at similar levels in all cell types (FIG. 1C). We noted that IRF7 was expressed at lower levels in CD4+ T cells, and at high levels in pDC as expected, which has been attributed to its ability to produce a strong IFNα response²⁷, that we extend here to IFNλ1 and IFNβ. It has been previously proposed that T cell receptor (TCR) stimulation is required to detect IFN-I expression following STING stimulation of murine CD4+ T cells²⁸. Stimulation of TCR-activated cells with ADU-S100 induced IFN-III but no detectable IFN-I (FIG. 1D), while donor-matched monocyte-derived dendritic cells (MDDC) produced both a 10-fold more IFNλ1 and high levels of IFNβ. Therefore, TCR stimulation is not sufficient to enable IFN-I in human CD4+ T cells.

DCs might be more efficient at taking up the synthetic ligands than CD4+ T cells. Therefore, we forced STING ligand entry from the media into cells using a well-established digitonin-mediated membrane permeabilization protocol through the rest of the study²⁹. Using this method, we found that CD4+ T cells remained 10- to 100-fold less capable of producing IFNλ1 mRNA and protein than MDDC, and IFNβ protein remained undetectable in CD4+ T cells (FIG. 1F). The phosphorylated levels of STING, TBK1 and IRF3 were identical between CD4+ T cells and MDDC in this assay. While we do not exclude kinetic signaling differences between the cell types, these results indicate that there is no general defect in activation of these key signaling proteins by cGAMP in CD4+ T cells (FIG. 1G).

We also asked if different ex vivo culture conditions between the donor-matched CD4+T cells and MDDC could impact the IFN-I/III response. To test this possibility, STING stimulation was carried out in activated CD4+ T cells exposed to MDDC-conditioned media or in MDDC exposed to CD4+ T cell-conditioned media. The IFN-I and IFN-III responses to cGAMP in CD4+ T cells did not change, while they further increased in MDDC exposed to CD4+T conditioned media (FIG. 1H). Type II IFNγ is produced by activated CD4+ T cells and we found that treatment of MDDC with IFNγ was sufficient to enhance their IFN-I/III response to cGAMP. This indicates that the environment of CD4+ T cells does not contain suppressive but rather stimulatory signals for IFN-I/III production in other cells, indicative of a cell-intrinsic restriction in CD4+ T cells.

To identify intrinsic factors that could regulate the IFN-I/III response of T cells in response to cGAMP, we performed a RNAseq analysis. This revealed an enrichment of antiviral and IFN-I related gene sets in the upregulated genes, indicating that the low IFN-I/III production by CD4+ T cells is biologically active (data not shown). The transcripts for IFNL1, IFNL2, IFNL3 and IFNB were detected, but counts were low relative to other transcripts. We did not detect changes induced by cGAMP for key signaling components or IFN receptor genes, except the IFN-stimulated gene IRF7 expression that was induced by cGAMP. To test if IRF7 induction could be sufficient to lift the restriction of IFN-I/III production in CD4+ T cells, we treated cells with IFNα2a treatment prior to cGAMP stimulation in CD4+ T cells. This treatment led to an increase in IFNα2 production by CD4+ T cells in response to high doses of cGAMP, but no induction of IFNβ and no significant increase of IFNλ1. Therefore, while increasing IRF7 can have a positive impact, it does not lift the restriction.

To determine if the IFN-I/III restriction was specific to STING activation, activated CD4+

T cells were infected with Sendai virus sensed by the RIG-1-MAVS pathway. Sendai virus induced both IFN-III and IFN-I in T cells, however the magnitude was 10- to 100-fold lower in comparison to donor-matched MDDC expressing similar levels of viral proteins (data not shown) These results indicate that human CD4+ T cells have the potential to produce IFN-I/III, but that it is restricted in response to external stimuli compared to DCs. This restriction occurs downstream of the phosphorylation events of STING-TBK1-IRF3 proteins.

### RELA is a Key Regulator of IFN Expression in CD4+ T Cells (FIG. 2)

We hypothesized that the regulation or availability of transcription factors could be restricting IFN expression in CD4+ T cells. In model systems, over-expression of NF-κB subunits is sufficient to induce the IFN-β promoter³⁰. Therefore, we increased the availability of RELA, a key NF-κB subunit that directly binds DNA implicated in IFN-I expression, through overexpression in CD4+ T cells and MDDC³¹. However, simply increasing RELA in CD4+ T cells and MDDC did not significantly enhance the IFN-I/III response to cGAMP neither at RNA nor protein levels, although there was a tendency for increased IFNλ1 in CD4+ T cells (FIG. 2B). These results suggested that RELA might be regulated in T cells to limit IFN promoter induction at the post-translational level. PTM of lysine residues in RELA such as acetylation and methylation are reported to impact transcriptional activity ^{32,33}. We focused on two RELA mutants converting five lysine residues distributed in the REL homology domain (K122, K123, K310, K314, K315) either to glutamine (RELA K5Q) or arginine (RELA K5R). The RELA K5Q is predicted to mimic acetylation while RELA K5R is predicted to maintain a non-acetylated basic state and both are reported to impact TNFα and IL1α driven gene expression^{32,34,35}. Overexpression of RELA, RELA K5R or RELA K5Q had no impact on endogenous RELA expression and they were expressed at similar levels in CD4+ T cells and MDDC (FIG. 2C). The IFN-I/III response to cGAMP in MDDC remained unchanged following RELA, RELA K5Q and RELA K5R overexpression compared to the control condition (FIG. 2D). In contrast, in CD4+ T cells, the non-acetylated RELA K5R increased baseline IFNλ1 expression and this response was augmented in response to cGAMP (FIG. 2D). Notably, IFNα2 was now detectable in response to cGAMP when CD4+ T cells expressed RELA K5R. This increase in IFNλ1 at baseline upon RELA K5R expression was not observed in MDDC, monocyte-derived macrophages (MDM) or THP-1 cells (data not shown). Furthermore, we noticed that the non-significant trend of increased IFNλ1 by RELA after cGAMP stimulation was abrogated by the acetylation mimic RELA K5Q. Together this data suggests that RELA regulation in activated CD4+ T cells differs from monocyte-derived cells in a manner that negatively impacts IFN-I/III expression. Notably, the lysine residues K122, 123, 310, 314 and 315 in RELA are important in determining the magnitude of IFN expression specifically in CD4+ T cells. K310 has been individually identified to modulate transcriptional activity of RELA and can also impact the PTM of surrounding lysine residues³⁶. This raised the possibility that K310 might be non-redundant among the 5 lysine residues responsible for controlling IFN-I/III expression in CD4+ T cells. To determine its importance, IFN response to cGAMP was compared between RELA K310R and RELA K5R in CD4+ T cells. K310R overexpression in CD4+ T cells increased baseline IFNλ1 similar to RELA K5R and the response was augmented in the presence of cGAMP (FIG. 2E, 2F). IFNα2 was also detected in K310R overexpressing cells stimulated with cGAMP, similar to RELA K5R overexpressing cells. This data highlights the role of K310 in RELA in tuning not only cGAMP-driven, but also tonic, IFN-I/III expression in CD4+ T cells.

IRF3 and DMNTi Synergize with RELA K5R to Unlock IFN-I/III Production by CD4+ T Cells (FIG. 3)

Since RELA K5R partially unlocks the restriction to IFN-I/III production in a T-cell specific manner, we asked whether additional factors that generally control IFN-I/III production could synergize to fully unlock the system.

We first over-expressed IRF3 alone in CD4+ T cells and MDDC. IRF3 over-expression resulted in a massive increase in phospho-IRF3 levels in response to cGAMP, that were equivalent in CD4+ T cells and MDDC, and other components of the pathway remained unchanged (FIG. 3C). Increasing IRF3 augmented cGAMP-mediated IFNλ1, IFNα2 and IFNβ production by 10-fold in MDDC. In contrast, IRF3 increased the IFNα2 response only to a small extent in CD4+ T cells and IFNβ remained undetectable (FIG. 3B). IFNL1 and IFNB transcripts were increased by IRF3 in CD4+ T cells in response to cGAMP (data not shown), indicating the promoters were responsive, but insufficiently. These results show that IRF3 is a limiting factor for IFN-I/III production, but its full potential observed in MDDC is restricted in CD4+ T cells. IFN expression is also subject to epigenetic regulation by histone modifications, DNA methylation and acetyl transferases³⁷⁻⁴⁰. A counter-intuitive feature of IFN and IFN-stimulated gene expression is a requirement for histone deacetylase (HDAC) activity, normally associated with gene expression silencing³⁸. To identify the dominant class of epigenetic regulation in CD4+ T cells, the IFN response to cGAMP was screened in the context of histone deacetylase inhibition (HDACi) by Trichostatin A (TSA) and DNA methylation inhibition (DNMTi) by 5′azacytidine (5AZA). TSA reduced the already low IFNλ1 production in CD4+ T cells. cGAMP-mediated STING signaling was intact in TSA-treated cells, consistent with an effect at the transcriptional level. In contrast, 5AZA enhanced the IFNλ1 response to cGAMP (FIG. 3D). This enhancement did not occur through detectable increase in STING signaling, in accordance with a transcriptional effect (FIG. 3E). These results revealed that IRF3 and 5AZA can enhance IFN-I/III expression in response to cGAMP stimulation, but not at baseline like RELA K5R. Next, we combined IRF3 and AZA with RELA K5R to assess synergy in IFN-I/III at the basal level and after cGAMP stimulation. Combining RELA K5R, IRF3 and 5AZA gradually increased the production of all tested IFN-I/III in CD4+ T cells in response to cGAMP (FIG. 3F). IFNλ1 was produced at similar levels to the MDDC benchmark in response to cGAMP (FIG. 3G). IFNα2a was also induced by more than 100-fold by CD4+ T cells, while it remained undetectable in MDDC (FIG. 3G). This difference could be explained by difference in levels of IRF7 between MDDC and CD4+ T cells in these conditions. IFNβ reached significant levels of production in CD4+ T cells, although they remain below MDDC (FIG. 3G). The expression of endogenous and overexpressed proteins was similar in all conditions evaluated. As control, we verified using reverse transcriptase inhibitors azidothymidine (AZT) and nevirapine (NVP) that the synergistic effects of IRF3 and RELA K5R expression in CD4+ T cells were specifically due to the lentivirus-mediated over-expression, and not to confounding factors associated with the viral lentivirus particles in the first place, such as cGAMP transferred in viral particles (data not shown)^{41,42}. Altogether, these results establish that the combination of RELA K5R, IRF3 and 5AZA in CD4+ T cells, which will be referred to as type I/III-interferon producing CD4+ T cells (iCD4+ T cells) in analogy to plasmacytoid dendritic cells ⁴³, unlocks their ability to produce type I and type 11 IFN to the same level as the dendritic cell benchmark in response to cGAMP.

An IRF7 Positive Feedback Drives IFN-I/III Production in iCD4+ T Cells (FIG. 4)

The enhanced expression of IRF7 in iCD4+T by RELA K5R suggested that a positive feedback from IFN-1 signaling might be important for IFN-1 production in these cells. To test this, CD4+ T cells were treated with decoy IFN-1 receptor B18R protein prior to cGAMP treatment 44. Adding B18R to iCD4+ T cells blocked cGAMP-dependent IFN-1 response (FIG. 4A). The IFNλ1 response was not strongly impacted by B18R exposure. To test if IRF7 was required for the positive feedback loop of IFN-I, IRF7 was knocked down using two different shRNA in the context of CD4+ T cells expressing only RELA K5R. As the basal level of IRF7 is low in CD4+ T cells, IFNα2a was added to the culture media to visualize IRF7 expression. This allowed a validation of IRF7 knock-down in CD4+ T cells (FIG. 4B). Both shRNA reduced IRF7 expression, and shRNA 1 was more potent (FIG. 4B). shRNA 1 abolished the induction of IFNα2 by cGAMP stimulation (FIG. 4C). The IRF7 shRNAs also reduced the production of IFNλ1 at baseline and after cGAMP stimulation. Notably, in the absence of RELA K5R, increasing IRF7 was not sufficient to enhance the IFN-I/III response in CD4+ T cells. Therefore, IRF7 mediates positive feedback to enable IFN-1 and maximize IFN-III production by iCD4+ T cells.

cGAS Drives the IRF7 Positive Feedback in iCD4+ T Cells (FIG. 5)

The expression of RELA K5R in CD4+ T cells was consistently sufficient to induce expression of IFNλ1 and IRF7. This raised the possibility that an upstream innate sensor could be active in CD4+ T cells to activate RELA K5R. We first tested the functionality of cGAS in T cells. cGAS over-expression is sufficient for cGAMP production due to promiscuous detection of endogenous nucleic acids, in particular in the nucleus 4. CD4+ T cells contained a low but detectable quantity of endogenous cGAMP. Over-expression of cGAS or nuclear localization signal NLS-cGAS in CD4+ T cells increased intracellular cGAMP levels by 10-20 fold. IFN-I/III remained undetectable (data not shown). We next examined the functionality of endogenous cGAS and other DNA sensors by nucleofecting a plasmid DNA coding for GFP. Intracellular cGAMP increased by 50-fold and minimal levels of IFNλ1 were induced by DNA transfection, while IFN-1 remained undetected. Therefore, endogenous cGAS is functional in T cells but not sufficient, even after over-expression, to unlock IFN-I/III expression, consistent with a downstream bottleneck at the level of RELA. Next, we evaluated the role of cGAS in the context of iCD4+ T cells. Knocking-out cGAS had no impact on IRF3 or RELA expression (FIG. 5A). However, cGAS KO iCD4+ T cells were unable to upregulate IRF7 (FIG. 5A). cGAS KO also inhibited the basal production of IFNλ1 and the IFNα2 response to cGAMP (FIG. 5C). To evaluate if cGAS activity was enhanced in cells expressing RELA K5R or IRF3, cellular cGAMP was quantified, but no significant increase was observed (FIG. 5B). cGAS localization determines its ability to be activated by endogenous DNA ligands 5,46,47. In activated CD4+ T cells, we found that cGAS is predominantly nuclear and this remained unchanged in cells overexpressing IRF3 or RELA K5R (FIG. 5D, 5E). These results show that tonic cGAS activation by endogenous ligands is required to drive the IRF7 loop leading to IFN-I/III expression in iCD4+ T cells.

iCD4+ T Cells Autonomously Resist HIV Infection (FIG. 6)

We next aimed to assess the functional impact of endowing CD4+ T cells with strong IFN-I/III responses. CD4+ T cells are the primary targets of HIV infection. CD4+ T cells are unable to mount an effective antiviral IFN-I response following infection, and therefore fail to protect themselves 7. We tested if iCD4+ T cells could now autonomously resist HIV-1 infection. Activated CD4+ T cells transduced with either control, IRF3, RELA K5R, IRF3 and RELA K5R in the presence of 5AZA were challenged with single round HIV-1 and HIV-2 reporter viruses. After 48 hours, infection rates and IFN levels were quantified. The rates of HIV-1 and HIV-2 infections were the lowest in iCD4+ T cells (IRF3+ RELA K5R+5AZA) (FIG. 6B). Notably, the levels of infection negatively correlated with baseline IFNλ1 levels detected across conditions (FIG. 6C). There was no further detectable increase in IFN-I/III response upon infection. This data provides evidence for the superior self-defense of iCD4+ T cells in the context of HIV infection.

### RELA K5R Increases the Anti-Tumor Activity of CAR T Cells

To extend these findings, we moved to anti-tumor chimeric antigen receptor T (CAR) cells.

A transient induction of IRF7-dependent IFN induction has been previously shown to be required for the optimal anti-tumor activity of CD19-targetting CAR T cells⁴⁸. Since IFN-I/III enhancement by RELA K5R was dependent on IRF7, we evaluated if RELA K5R could improve CART mediated tumor killing using a spheroid tumor model of A549 cells expressing CD19. T cells transduced with CD19-targeting CART with control GFP or RELA K5R were added to tumors and tumor growth was monitored for 5-6 days. Since the rate of CAR transduction varied slightly when co-transduced with GFP or RELA K5R vectors, the cell numbers were adjusted to normalize the CAR-positive cells. The spheroids grew in size with time when cultured alone and reduced in size partially when co-cultured with CD19-targeting CAR T cells (FIG. 6C, 6D). When CAR T cells co-expressed RELA K5R, tumor cells were significantly more efficiently eliminated when compared to CAR T cells alone (FIG. 6E, 6F). Notably, RELA K5R delay of tumor growth was also observed with lower numbers of CAR T cells (FIG. 6F, left panel). IFNL1 was detected at the end of the 6 days co-culture in all dilutions of CART and RELA K5R cells. The improved killing capacity of RELA K5R was not due to increased proliferation rates (data not shown). Altogether, these results demonstrate that RELA K5R improves the anti-tumor activity of CAR T cells.

### DISCUSSION

In this study, we addressed the functionality of cGAS-STING-IFN innate sensing pathway in human CD4+ T cells. We identified that the production of IFN-III and to a much larger extent IFN-I is tightly regulated in CD4+ T cells with several restrictions at the level of both transcription (RELA, IRF3) and epigenetic factors (DNA methylation). To understand the basis of low IFN-I/III responses, we systematically compared the IFN-I/III response of CD4+ T cells and dendritic cells. We find that upstream phosphorylation-based signaling by cGAS-STING-TBK1-IRF3 is intact in CD4+ T cells. Instead, we identify RELA as a limiting factor of both tonic and inducible IFN-I/III expression in T cells. The crucial role of RELA is further supported by our finding that five lysines (122, 123, 310, 314 and 315) are key determinants of IFN-I/III expression in CD4+ T cells, but not in dendritic cells. IRF3 expression and DNA methylation inhibition synergize with RELA K5R to enhance IFN expression, but unlike RELA K5R, they are not able to promote spontaneous IFN-I/III expression. Therefore, we propose that RELA is a rheostat for IFN-I/III expression in lymphocytes 49. The PTM of each of the five lysines of RELA have been reported to have variable effects depending on the target gene and the stimulus. We used previous reported mutations to address the role of these lysines. Mutations to acetyl-mimic glutamine blunted the enhancement of IFNλ1 by RELA, while mutations to arginine, which mimic a lack of PTM, enhanced IFN-I/III production. Both RELA K5R and RELA K5Q mutations have been previously shown to induce p65-dependent genes in response to stimuli such as TNFα and IL1α^{50,51}. However, the overlap of differentially expressed genes is low suggesting differential affinity for co-factors rather than DNA binding affinity or transactivation potential ^{50,51}. In T cells, RELA is activated downstream of the TCR but the PTM state of these lysines has not been extensively studied, with the exception of K310. K310 is reported to be acetylated by p300 in response to TCR stimulation and subsequently deacetylated by SIRT1 to avoid hyperactivation⁵²⁻⁵⁵. In other cell types, while acetylation of RELA at these residues has been associated with both activation and repression of gene expression^{51,56,57}, methylation is reported to negatively impact NF-κB-mediated gene expression⁵⁸⁻⁶⁰. Further studies are required to appreciate the roles of the various RELA PTM in the control of IFN-I/III production by T cells. Interestingly, RELA in MDDC, MDM and THP-1 cells did not appear to be a limiting factor with neither RELA lysine mutants impacting IFN-I/III expression at baseline or in response to cGAMP. It is thus tempting to speculate that the pool of endogenous RELA in T cells may be preferentially associated with T-cell specific gene programs, such as TCR signaling-induced genes, as opposed to PRR-induced IFN-I/III genes.

We found that unlike RELA, overexpression of IRF3 enhances IFN-I expression in both MDDC and CD4+ T cells, suggesting that IRF3 is a limiting factor in both cell types tested. We also demonstrate that IRF7 induction by RELA is crucial for inducing IFN-I and enhancing IFN-III expression in T cells. Importantly however, IRF3 and IRF7 are individually insufficient to promote IFN-III and IFN-I expression in T cells. We also show that treatment of T cells with 5′AZA has a positive impact of IFN expression in T cells. A recent study identified a single cytosine in the IFNB promoter that, when methylated, negatively impacts IRF3 recruitment and thus IFN expression in murine macrophages⁶¹. Alternatively, inhibition of DNA methyltransferases could indirectly enhance IFN-I expression through re-expression of endogenous retroelements and activation of innate sensors³⁷. We also found that TSA inhibits IFN expression at baseline with RELA K5R and after cGAMP stimulation. HDAC inhibition has been shown to reactivate HIV expression from latent integrated proviruses in vitro and advanced to clinical trials in vivo⁶². We propose that in addition to activating the viral promoters, HDAC inhibition also diminishes IFN expression in vivo, thereby promoting viral expression. Altogether, our findings highlight an emerging regulation of IFN gene expression by epigenetic regulation.

We describe tonic IFN production in T cells that is dependent on cGAS, RELA and IRF7 expression. In the assays we used, baseline IFN production is detectable at the protein level for IFNλ1 when unlocked by RELA K5R in T cells, but IFN-I remains below detection limits in accordance with tonic signaling². The substrate responsible for cGAS activity is unclear, but we find that cGAS is largely nuclear in T cells and its association with nuclear DNA could permit low levels of cGAS activity as described in other cells⁶³. Notably neither the activity nor cellular localization of cGAS is altered upon RELA K5R overexpression.

Consistent with restriction of IFN-I/III in T cells, human T cells have not been previously identified as a cellular source of IFN-I or IFN-III in patients with interferonopathies⁶⁴. Intriguingly, patients carrying mutations in RELA have been described to have elevated IFN-I and ISG signature at steady state⁶⁵. However, the mechanism of how these RELA mutants might induce unchecked IFN-I expression, the requirement for upstream intracellular sensors and cellular subtypes involved in the IFN-I induction in the pathology of these patients is currently unclear.

We provide evidence that intrinsic induction of IFN-I/III in T cells can be beneficial in two contexts. First, it increases resistance against HIV infection. Second, RELA K5R is able to improve tumor elimination by CAR T cells robustly in vitro, leading to the relevancy of using RELA K5R CAR+ T cells in therapy. Notably, given that RELA lies downstream of several receptors, the impact of RELA K5R may be broader than IFN-I/III expression in T cells. Therefore, RELA emerges as a target for enhancing IFN-I/III expression by T cells in these therapeutic contexts.

Our findings show that T cells, unlike antigen-presenting cells, are specifically programmed to limit IFN-I/III expression, even upon PRR activation. This implies that the ability of CD4+ T cells to produce IFN-I/III production has been counter-selected during evolution. In contrast to T cells, pDC are well appreciated as a major producer of IFN-I in infections and in pathological conditions, and they constitute a minor fraction of circulating and tissue-resident immune cells. We thus propose that the restriction of IFN-I/III expression in T cells is a mechanism to avoid pathogenic levels of a toxic cytokine from a cell type that constitutes a large proportion of immune cells.

### BIBLIOGRAPHIC DATA

1. Iwasaki, A. & Medzhitov, R. Regulation of adaptive immunity by the innate immune system. Science 327, 291-295 (2010).
2. Gough, D. J., Messina, N. L., Clarke, C. J., Johnstone, R. W. & Levy, D. E. Constitutive type I interferon modulates homeostatic balance through tonic signaling. Immunity 36, 166-74 (2012).
3. Schoggins, J. W. et al. Pan-viral specificity of IFN-induced genes reveals new roles for cGAS in innate immunity. Nature 505, 691-5 (2014).
4. Hartlova, A. et al. DNA damage primes the type I interferon system via the cytosolic DNA sensor STING to promote anti-microbial innate immunity. Immunity 42, 332-43 (2015).
5. Gentili, M. et al. The N-Terminal Domain of cGAS Determines Preferential Association with Centromeric DNA and Innate Immune Activation in the Nucleus. Cell Rep 26, 2377-2393.e13 (2019).
6. Basagoudanavar, S. H. et al. Distinct Roles for the NF-κB ReIA Subunit during Antiviral Innate Immune Responses. J. Virol. 85, 2599-2610 (2011).
7. Manel, N. et al. A cryptic sensor for HIV-1 activates antiviral innate immunity in dendritic cells. Nature 467, 214-217 (2010).
8. Eisner, C. et al. Absence of cGAS-mediated type I IFN responses in HIV-1-infected T cells. Proc. Natl. Acad. Sci. U.S.A 117, 19475-19486 (2020).
9. Angin, M. et al. Metabolic plasticity of HIV-specific CD8+ T cells is associated with enhanced antiviral potential and natural control of HIV-1 infection. Nat. Metab. 1, 704-716 (2019).
10. Zhao, Z. et al. Structural Design of Engineered Costimulation Determines Tumor Rejection Kinetics and Persistence of CAR T Cells. Cancer Cell 28, 415-428 (2015).
11. Cerboni, S. et al. Intrinsic antiproliferative activity of the innate sensor STING in T lymphocytes. J. Exp. Med. 214, 1769-1785 (2017).
12. Li, P. et al. Stimulating the RIG-1 pathway to kill cells in the latent HIV reservoir following viral reactivation. Nat. Med. 22, 807-811 (2016).
13. Berg, R. K. et al. T cells detect intracellular DNA but fail to induce type I IFN responses: implications for restriction of HIV replication. PloS One 9, e84513 (2014).
14. Simpson, S. R. et al. T Cells Produce IFN-α in the TREX1 D18N Model of Lupus-like Autoimmunity. J. Immunol. (2019) doi:10.4049/jimmunol.1900220.
15. Kagamu, H. et al. CD4+ T-cell Immunity in the Peripheral Blood Correlates with Response to Anti-PD-1 Therapy. Cancer Immunol. Res. 8, 334-344 (2020).
16. Chen, J. et al. Reprogramming immunosuppressive myeloid cells by activated T cells promotes the response to anti-PD-1 therapy in colorectal cancer. Signal Transduct. Target. Ther. 6, 4 (2021).
17. Li, W. et al. cGAS-STING-mediated DNA sensing maintains CD8+ T cell stemness and promotes antitumor T cell therapy. Sci. Transl. Med. 12, eaay9013 (2020).
18. Gulen, M. F. et al. Signalling strength determines proapoptotic functions of STING. Nat. Commun. 8, 427 (2017).
19. Wu, J. et al. STING-mediated disruption of calcium homeostasis chronically activates ER stress and primes T cell death. J. Exp. Med. 216, 867-883 (2019).
20. Conlon, J. et al. Mouse, but not human STING, binds and signals in response to the vascular disrupting agent DMXAA. J. Immunol. Baltim. Md 1950 190, 5216-5225 (2013).
21. Bouis, D. et al. Severe combined immunodeficiency in stimulator of interferon genes (STING) V154M/wild-type mice. J. Allergy Clin. Immunol. 143, 712-725.e5 (2019).
22. Luksch, H. et al. STING-associated lung disease in mice relies on T cells but not type I interferon. J. Allergy Clin. Immunol. 144, 254-266.e8 (2019).
23. Liu, Y. et al. Activated STING in a vascular and pulmonary syndrome. N. Engl. J. Med. 371, 507-518 (2014).
24. Jeremiah, N. et al. Inherited STING-activating mutation underlies a familial inflammatory syndrome with lupus-like manifestations. J. Clin. Invest. 124, 5516-5520 (2014).
25. Cerboni, S. et al. Intrinsic antiproliferative activity of the innate sensor STING in T lymphocytes. J. Exp. Med. 214, 1769-1785 (2017).
26. Corrales, L. et al. Direct Activation of STING in the Tumor Microenvironment Leads to Potent and Systemic Tumor Regression and Immunity. Cell Rep 11, 1018-30 (2015).
27. Honda, K. et al. IRF-7 is the master regulator of type-I interferon-dependent immune responses. Nature 434, 772-777 (2005).
28. Imanishi, T. et al. Reciprocal regulation of STING and TCR signaling by mTORC1 for T-cell activation and function. Life Sci. Alliance 2, e201800282 (2019).
29. Woodward, J. J., lavarone, A. T. & Portnoy, D. A. c-di-AMP Secreted by Intracellular Listeria monocytogenes Activates a Host Type I Interferon Response. Science (2010) doi:10.1126/science.1189801.
30. Apostolou, E. & Thanos, D. Virus Infection Induces NF-κB-Dependent Interchromosomal Associations Mediating Monoallelic IFN-β Gene Expression. Cell 134, 85-96 (2008).
31. Freaney, J. E., Kim, R., Mandhana, R. & Horvath, C. M. Extensive Collaboration of Immune Master Regulators IRF3 and NFκB in RNA Pol II Recruitment and Pause-Release in Human Innate Antiviral Transcription. Cell Rep. 4, 959-973 (2013).
32. Li, H. et al. Regulation of NF-κB activity by competition between ReIA acetylation and ubiquitination. Oncogene 31, 611-623 (2012).
33. Chen, L., Mu, Y. & Greene, W. C. Acetylation of ReIA at discrete sites regulates distinct nuclear functions of NF-kappaB. EMBO J. 21, 6539-6548 (2002).
34. Ziesché, E. et al. The coactivator role of histone deacetylase 3 in IL-1-signaling involves deacetylation of p65 NF-κB. Nucleic Acids Res. 41, 90-109 (2013).
35. Buerki, C. et al. Functional relevance of novel p300-mediated lysine 314 and 315 acetylation of ReIA/p65. Nucleic Acids Res. 36, 1665-1680 (2008).
36. Yang, X.-D., Tajkhorshid, E. & Chen, L.-F. Functional interplay between acetylation and methylation of the ReIA subunit of NF-kappaB. Mol. Cell. Biol. 30, 2170-2180 (2010).
37. Chiappinelli, K. B. et al. Inhibiting DNA Methylation Causes an Interferon Response in Cancer via dsRNA Including Endogenous Retroviruses. Cell 162, 974-986 (2015).
38. Marié, I. J., Chang, H.-M. & Levy, D. E. HDAC stimulates gene expression through BRD4 availability in response to IFN and in interferonopathies. J. Exp. Med. 215, 3194-3212 (2018).
39. Salvi, V. et al. Trichostatin A blocks type I interferon production by activated plasmacytoid dendritic cells. Immunobiology 215, 756-761 (2010).
40. Chen, X. et al. Requirement for the histone deacetylase Hdac3 for the inflammatory gene expression program in macrophages. Proc. Natl. Acad. Sci. U.S.A 109, E2865-2874 (2012).
41. Gentili, M. et al. Transmission of innate immune signaling by packaging of cGAMP in viral particles. Science 349, 1232-1236 (2015).
42. Bridgeman, A. et al. Viruses transfer the antiviral second messenger cGAMP between cells. Science 349, 1228-1232 (2015).
43. Soumelis, V. & Liu, Y.-J. From plasmacytoid to dendritic cell: morphological and functional switches during plasmacytoid pre-dendritic cell differentiation. Eur. J. Immunol. 36, 2286-2292 (2006).
44. Herniez, B. et al. A virus-encoded type I interferon decoy receptor enables evasion of host immunity through cell-surface binding. Nat. Commun. 9, 5440 (2018).
45. Gentili, M. et al. The N-Terminal Domain of cGAS Determines Preferential Association with Centromeric DNA and Innate Immune Activation in the Nucleus. Cell Rep. 26, 2377-2393.e13 (2019).
46. Li, T. et al. Phosphorylation and chromatin tethering prevent cGAS activation during mitosis. Science 371, eabc5386 (2021).
47. Barnett, K. C. et al. Phosphoinositide Interactions Position cGAS at the Plasma Membrane to Ensure Efficient Distinction between Self- and Viral DNA. Cell 176, 1432-1446 e11 (2019).
48. Zhao, Z. et al. Structural Design of Engineered Costimulation Determines Tumor Rejection Kinetics and Persistence of CAR T Cells. Cancer Cell 28, 415-428 (2015).
49. Bardwell, L. Signal Transduction: Turning a Switch into a Rheostat. Curr. Biol. 18, R910-R912 (2008).
50. Buerki, C. et al. Functional relevance of novel p300-mediated lysine 314 and 315 acetylation of ReIA/p65. Nucleic Acids Res. 36, 1665-1680 (2008).
51. Li, H. et al. Regulation of NF-κB activity by competition between ReIA acetylation and ubiquitination. Oncogene 31, 611-623 (2012).
52. Clavijo, P. E. & Frauwirth, K. A. Anergic CD8+T Lymphocytes Have Impaired NF-κB Activation with Defects in p65 Phosphorylation and Acetylation. J. Immunol. 188, 1213-1221 (2012).
53. Kwon, H.-S. et al. Human immunodeficiency virus type 1 Tat protein inhibits the SIRT1 deacetylase and induces T cell hyperactivation. Cell Host Microbe 3, 158-167 (2008).
54. Yeung, F. et al. Modulation of NF-kappaB-dependent transcription and cell survival by the SIRT1 deacetylase. EMBO J. 23, 2369-2380 (2004).
55. Zhang, J. et al. The type III histone deacetylase Sirt1 is essential for maintenance of T cell tolerance in mice. J. Clin. Invest. 119, 3048-3058 (2009).
56. Kiernan, R. et al. Post-activation turn-off of NF-kappa B-dependent transcription is regulated by acetylation of p65. J. Biol. Chem. 278, 2758-2766 (2003).
57. Ziesché, E. et al. The coactivator role of histone deacetylase 3 in IL-1-signaling involves deacetylation of p65 NF-κB. Nucleic Acids Res. 41, 90-109 (2013).
58. Levy, D. et al. Lysine methylation of the NF-κB subunit ReIA by SETD6 couples activity of the histone methyltransferase GLP at chromatin to tonic repression of NF-κB signaling. Nat. Immunol. 12, 29-36 (2011).
59. Yang, X.-D. et al. Negative regulation of NF-kappaB action by Set9-mediated lysine methylation of the ReIA subunit. EMBO J. 28, 1055-1066 (2009).
60. Yang, X.-D., Tajkhorshid, E. & Chen, L.-F. Functional interplay between acetylation and methylation of the ReIA subunit of NF-kappaB. Mol. Cell. Biol. 30, 2170-2180 (2010).
61. Nishioka, K., Daidoji, T. & Nakaya, T. Demethylation around the transcriptional start site of the IFN-β gene induces IFN-β production and protection against influenza virus infection. Biochem. Biophys. Res. Commun. 520, 269-276 (2019).
62. Lehrman, G. et al. Depletion of latent HIV-1 infection in vivo: a proof-of-concept study. The Lancet 366, 549-555 (2005).
63. Gentili, M. et al. The N-Terminal Domain of cGAS Determines Preferential Association with Centromeric DNA and Innate Immune Activation in the Nucleus. Cell Rep. 26, 2377-2393.e13 (2019).
64. Rodero, M. P. et al. Detection of interferon alpha protein reveals differential levels and cellular sources in disease. J. Exp. Med. 214, 1547-1555 (2017).
65. Barnabei, L. et al. Heterozygous RELA mutations cause early-onset systemic lupus erythematosus by hijacking the NF-κB pathway towards transcriptional activation of type-I Interferon genes. bioRxiv 2020.04.27.046102 (2020) doi:10.1101/2020.04.27.046102.
66. Ruffin, N. et al. Constitutive Siglec-1 expression confers susceptibility to HIV-1 infection of human dendritic cell precursors. Proc. Natl. Acad. Sci. U.S.A 116, 21685-21693 (2019).
67. Manel, N. et al. A cryptic sensor for HIV-1 activates antiviral innate immunity in dendritic cells. Nature 467, 214-217 (2010).
68. Döring, M. et al. Single-cell analysis reveals divergent responses of human dendritic cells to the MVA vaccine. Sci. Signal. 14, eabd9720 (2021).
69. Bhargava, A. et al. Inhibition of HIV infection by structural proteins of the inner nuclear membrane is associated with reduced chromatin dynamics. Cell Rep. 36, 109763 (2021).
70. Alcami, A. & Smith, G. L. A soluble receptor for interleukin-1 beta encoded by vaccinia virus: a novel mechanism of virus modulation of the host response to infection. Cell 71, 153-167 (1992).
71. Dobin, A. et al. STAR: ultrafast universal RNA-seq aligner. Bioinformatics 29, 15-21 (2013).
72. Wang, L., Wang, S. & Li, W. RSeQC: quality control of RNA-seq experiments. Bioinformatics 28, 2184-2185 (2012).
73. Love, M. I., Huber, W. & Anders, S. Moderated estimation of fold change and dispersion for RNA-seq data with DESeq2. Genome Biol. 15, 550 (2014).
74. Cerboni, S., Marques-Ladeira, S. & Manel, N. Virus-stimulated Dendritic Cells Elicit a T Antiviral Transcriptional Signature in Human CD4+ Lymphocytes. J. Mol. Biol. 167389 (2021) doi:10.1016/j.jmb.2021.167389.
75. Silvin, A. et al. Constitutive resistance to viral infection in human CD141(+) dendritic cells. Sci Immunol 2, (2017).
76. Yu, G., Wang, L.-G., Han, Y. & He, Q.-Y. clusterProfiler: an R Package for Comparing Biological Themes Among Gene Clusters. OMICS J. Integr. Biol. 16, 284l-287(2012).
77. Schindelin, J. et al. Fiji: an open-source platform for biological-image analysis. Nat. Methods 9, 676-682 (2012).

1. An immune cell comprising and/or expressing a modified RELA protein derived from a wild-type RELA protein by substitution of at least one lysine residue for a non-lysine residue, the modified RELA protein having the ability to bind to a DNA sequence involved in the transcription of Interferon (IFN), the modified RELA protein eliciting INF production in the immune cell.

2. The immune cell according to claim 1, wherein the at least one substituted lysine residue is localized at position 122, 123, 310, 314 or 315, of the wild-type RELA protein.

3. The immune cell according to claim 1, wherein at least one substituted lysine residue is substituted for an arginine residue.

4. The immune cell according to claim 1, wherein at least one substituted lysine residue is localized at position 310 of the wild-type RELA protein, and is substituted for an arginine residue, or wherein the lysine residues localized at position 122, 123, 310, 314 and 315 of the wild-type RELA protein, are each substituted for an arginine residue.

5. The immune cell according to claim 1, which is a CD4+ T cell or a CD8+ T cell.

6. The immune cell according to claim 1, which is a T cell or a CAR-T cell, and which comprises or expresses a modified RELA protein having the amino acid sequence set forth in SEQ ID No. 2 or SEQ ID No. 3.

7. The immune cell according to claim 1, for use in the treatment of a NF-κB-associated disease such as autoimmune, inflammatory, and malignant disorders including multiple sclerosis, rheumatoid arthritis, atherosclerosis, inflammatory bowel diseases and malignant tumors.

8. The immune cell according to claim 1, for use in the treatment of a cancer or an infection.

9. A modified RELA protein, wherein the modified RELA protein is derived from a wild-type RELA protein, by substitution of at least one lysine residue for a non-lysine residue, the modified RELA protein having the ability to bind to a DNA sequence involved in the transcription of Interferon (IFN), for use in the elicitation of the production of interferon (IFN), in immune cells.

10. A modified RELA protein, wherein the modified RELA protein is derived from a wild-type RELA protein, by substitution of at least one lysine residue for a non-lysine residue, the modified RELA protein having the ability to bind to a DNA sequence involved in the transcription of Interferon (IFN), for use in the treatment of a of a NF-κB-associated disease, by the elicitation of the production of interferon (IFN), in immune cells.

11. The modified RELA protein according to claim 9 for the use according to claim 9, wherein the at least one substituted lysine residue is localized at position 122, 123, 310, 314 or 315, of the wild-type RELA protein.

12. The modified RELA protein according to claim 9 for the use according to claim 9, wherein at least one substituted lysine residue is substituted for an arginine residue.

13. The modified RELA protein according to claim 9 for the use according to claim 9, wherein at least one substituted lysine residue is localized at position 310 of the wild-type RELA protein, and is substituted for an arginine residue, or wherein the lysine residues localized at position 122, 123, 310, 314 and 315 of the wild-type RELA protein, are each substituted for an arginine residue.

14. The modified RELA protein according to claim 9 for the use in the treatment of a NF-κB-associated disease such as autoimmune, inflammatory, and malignant disorders including multiple sclerosis, rheumatoid arthritis, atherosclerosis, inflammatory bowel diseases and malignant tumors, or for the use in the treatment of a cancer or an infection.

15. The modified RELA protein according to claim 9, for the use according to claim 9, wherein the modified RELA protein is expressed in an immune cell.

16. A genetic construct, encoding a modified RELA protein according to claim 9, for use in the engineering of an immune cell.

17. A method for providing engineered immune cells, with an enhanced interferon (IFN) metabolism, the method comprising the following step:
Expressing within immune cells a modified RELA protein according to claim 14 to provide engineered immune cells, or introducing within immune cells the genetic construct according to claim 14,
Optionally contacting the (engineered) immune cells with a DNA methylation inhibitor, and/or overexpressing IRF3 in the (engineered) immune cells,
Selecting immune cells that produces more interferon, in particular IFN-I and/or IFN-III, than non-engineered immune cells.


SEQUENCE LISTING (29 sequences)

SEQ ID NO: 1 - protein, 551 aa, synthetic construct
  REGION 1..551: note = synthetic sequence
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 2 - protein, 551 aa, synthetic construct
  REGION 1..551: note = synthetic sequence
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFRSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 3 - protein, 551 aa, synthetic construct
  REGION 1..551: note = synthetic sequence
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVRRRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFRSIMRRSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 4 - protein, 551 aa, synthetic construct
  REGION 1..551: note = synthetic sequence
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVRKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFRSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 5 - protein, 551 aa, synthetic construct
  REGION 1..551: note = synthetic sequence
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKRRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFRSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 6 - protein, 551 aa, synthetic construct
  REGION 1..551: note = synthetic sequence
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFRSIMRKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 7 - protein, 551 aa, synthetic construct
  REGION 1..551: note = synthetic sequence
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFRSIMKRSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 8 - protein, 551 aa, synthetic construct
  REGION 1..551: note = synthetic sequence
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVRRRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFRSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 9 - protein, 551 aa, synthetic construct
  REGION 1..551: note = synthetic sequence
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVRKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFRSIMRKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 10 - protein, 551 aa, synthetic construct
  REGION 1..551: note = synthetic sequence
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVRKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFRSIMKRSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 11 - protein, 551 aa, synthetic construct
  REGION 1..551: note = synthetic sequence
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKRRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFRSIMRKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 12 - protein, 551 aa, synthetic construct
  REGION 1..551: note = synthetic sequence
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKRRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFRSIMKRSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 13 - protein, 551 aa, synthetic construct
  REGION 1..551: note = synthetic sequence
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFRSIMRRSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 14 - protein, 551 aa, synthetic construct
  REGION 1..551: note = synthetic sequence
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVRRRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFRSIMRKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 15 - protein, 551 aa, synthetic construct
  REGION 1..551: note = synthetic sequence
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVRRRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFRSIMKRSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 16 - protein, 551 aa, synthetic construct
  REGION 1..551: note = synthetic sequence
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVRKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFRSIMRRSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 17 - protein, 551 aa, synthetic construct
  REGION 1..551: note = synthetic sequence
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKRRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFRSIMRRSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 18 - DNA, 21 bp, synthetic construct
  misc_feature 1..21: note = synthetic sequence
  source 1..21: mol_type = other DNA
cccgagctgcacgttcctata

SEQ ID NO: 19 - DNA, 21 bp, synthetic construct
  misc_feature 1..21: note = synthetic sequence
  source 1..21: mol_type = other DNA
cgcagcgtgagggtgtgtctt

SEQ ID NO: 20 - DNA, 20 bp, synthetic construct
  misc_feature 1..20: note = synthetic sequence
  source 1..20: mol_type = other DNA
agactcggtgggatccatcg

SEQ ID NO: 21 - DNA, 20 bp, synthetic construct
  misc_feature 1..20: note = synthetic sequence
  source 1..20: mol_type = other DNA
cgaagccaagacctccgccc

SEQ ID NO: 22 - DNA, 20 bp, synthetic construct
  misc_feature 1..20: note = synthetic sequence
  source 1..20: mol_type = other DNA
ggtgactttggtgctaggct

SEQ ID NO: 23 - DNA, 20 bp, synthetic construct
  misc_feature 1..20: note = synthetic sequence
  source 1..20: mol_type = other DNA
tgagtgactcttccaaggcg

SEQ ID NO: 24 - DNA, 20 bp, synthetic construct
  misc_feature 1..20: note = synthetic sequence
  source 1..20: mol_type = other DNA
gtctcctccaaattgctctc

SEQ ID NO: 25 - DNA, 20 bp, synthetic construct
  misc_feature 1..20: note = synthetic sequence
  source 1..20: mol_type = other DNA
acaggagcttctgacactga

SEQ ID NO: 26 - DNA, 20 bp, synthetic construct
  misc_feature 1..20: note = synthetic sequence
  source 1..20: mol_type = other DNA
ggacttcgagcaagagatgg

SEQ ID NO: 27 - DNA, 20 bp, synthetic construct
  misc_feature 1..20: note = synthetic sequence
  source 1..20: mol_type = other DNA
agcactgtgttggcgtacag

SEQ ID NO: 28 - DNA, 20 bp, synthetic construct
  misc_feature 1..20: note = synthetic sequence
  source 1..20: mol_type = other DNA
tctctgctggatgacgtgag

SEQ ID NO: 29 - DNA, 19 bp, synthetic construct
  misc_feature 1..19: note = synthetic sequence
  source 1..19: mol_type = other DNA
tagctgtgctcgcgctact